(19) United States
(12) Patent Application Publication
(10) Pub. No.: US 2002/0177829 A1
(43) Pub. Date: Nov. 28, 2002

(54) Disposable underpants and method

(76) Inventors: David Arthur Fell, Neenah, WI (US); Jacqueline Ann Gross, Neenah, WI (US); Joseph Patrick Fell, Appleton, WI (US); Amy Lynn Fletcher, Appleton, WI (US); Sarah Jane Marie Freiburger, Kaukauna, WI (US)

Correspondence Address: DOUGLAS G GLANTZ, ATTORNEY AT LAW, 5260 DEBORAH COURT, DOYLESTOWN, PA, 18901, US

(21) Appl. No.: 09/796,375

(22) Filed: Mar. 1, 2001

(51) Int. Cl. A61F 13/15; A61F 13/20

(52) U.S. Cl. 604/385.01; 604/385.25; 604/385.29; 604/385.3

(57) ABSTRACT

A three dimensional disposable underpant is disclosed having an absorbent core skewed forward by a factor of no more than 0.10. The absorbent core is positioned within the front, central, and back sections of the disposable underpant such that the length of the absorbent core in the back section of the disposable underpant divided by the length of the absorbent core in the front and central sections of the disposable underpant is less than 0.10, and the length of the absorbent core in the front section of the disposable underpant is greater than the length of the absorbent core in the back section of the disposable underpant. In one aspect, the disposable underpant includes elasticized leg and waist openings, resulting in the underpant being stretchable about the hip and stomach regions of a user.

### BACKGROUND OF THE INVENTION

1. Technical Field

This invention relates to disposable underpants. In one aspect, this invention relates to disposable underpants having a skewed absorbent core. In one aspect, this invention relates to disposable incontinence underpants having a liquid impervious portion and an absorbent core shifted forward for containing and absorbing body discharges and waste.

2. Background

Underpant absorbent garments for absorbing human body waste discharges include non-disposable underpants, non-disposable underpants in combination with incontinence articles or absorbent articles, and disposable underpants or disposable absorbent undergarments.

Disposable absorbent undergarments have a structure including an aqueous-liquid-pervious topsheet layer, an absorbent core containing one or more layers for receiving and absorbing a human body waste discharge, and an aqueous-liquid-impervious liquid barrier for containing the discharge.

The disposable underpant is a three-layer composite structure including a liquid-permeable bodyside inner liner, topsheet layer; a liquid-impermeable outer cover, liquid barrier; and an absorbent batt sandwiched between the inner liner and the outer cover. The conventional absorbent batt is placed symmetrically between a front waist band and a back waist band. Materials for the three principal elements of the disposable underpant include nonwoven fabrics for the bodyside inner liner, topsheet layer; a thin thermoplastic film for the outer cover, liquid barrier; and cellulosic fluff for the absorbent batt.

Disposable underpants provide adult incontinence care and infant care.

### INTRODUCTION TO THE INVENTION

Some absorbent garments perform satisfactorily for their intended purpose, but there remains a need to provide a more discrete underpant having preferred absorption characteristics, preferred utilization of absorbent material, and preferred waste containment characteristics with a minimum of discomfort to the wearer. Underpants have not served to facilitate the transfer of aqueous liquids to an entire area of an absorbent layer or layers, including the distal ends of the absorbent layer or layers. Waste absorption is concentrated in a small region of the absorbent layer which results in an under-utilization of much of the absorbent capacity of the undergarment.

Because of differences between small children and adult bodies and different activities and movements of small children and adults, increasing the size of disposable training pants to adult sizes does not meet many of the needs of adult users.

Absorbent underpants are worn in a “J” configuration. In a “J” configuration, the front region of the undergarment is worn lower on the wearer's body than the back region of the undergarment.

Absorbent underpants are worn on the wearer's body such that the center of the underpant does not coincide with the point of insult. The point of insult occurs toward the front region of the underpant. Accordingly, currently available underpants do not provide adequate absorbent material at the point of insult.

Absorbent underpants having absorbent pads including centrally located acquisition zones do not provide a preferred absorbency, resulting in product failure.

Some underpants for absorbing and containing human body waste discharge have been bulky and somewhat ineffective. The absorbent core is positioned to have an absorbent capacity location not fully utilized in a bulky configuration, particularly in the central portion and the back region. Such undergarments are uncomfortable to wear, especially if the wearer is an active adult. Such undergarments are costly and inefficient in placement of the absorbent material in the back region where it is not used, but rather wasted.

Thus, a need exists for an absorbent underpant having preferred absorbent characteristics and preferred containment characteristics while still being comfortable to wear.

Further objects of the present invention will become apparent from the detailed description which follows.

### SUMMARY OF THE INVENTION

The article and method of the present invention provide a disposable underpant defining an initial expanded shape having longitudinal and transverse axes, a front section, a back section, the front section and the back section being oppositely disposed on the longitudinal axis, and a central section disposed between the front section and the back section, each of the sections having a length along the longitudinal axis of one third of the length of the underpant. An outer cover having a front body portion and a back body portion are connected by a crotch portion, the front and back body portions being connected together to form a waist opening and two leg openings, and an absorbent barrier composite associated within at least the crotch portion has a liquid impervious layer, a pervious layer, and a rectangular absorbent core having a front end edge and a back end edge positioned between the liquid impervious layer and a pervious layer. The absorbent core is disposed within the sections such that the length of the absorbent core in the back section divided by the length of the absorbent core in the front section and the central section is less than 0.10, and the length of the absorbent core in the front section is greater than the length of absorbent core in the back section.

In one aspect, the outer cover further includes leg elastic positioned around each of the leg openings to form a gather around each leg opening, and a waist portion elastic positioned around the front and back body portions forms gathers of the front and back body portions.

### BRIEF DESCRIPTION OF THE DRAWINGS

FIG. 1a is a top plan view of a disposable underpant article of the present invention in a preassembled flat configuration.

FIG. 1b is a top plan view of a disposable underpant article of the present invention in a preassembled flat configuration.

FIG. 1c is a top plan view of a disposable underpant article of the present invention in a preassembled flat configuration.

FIG. 2a is a top plan view of a disposable underpant article of the present invention in a preassembled flat configuration.

FIG. 2b is a top plan view of a disposable underpant article of the present invention in a preassembled flat configuration.

FIG. 3 is a top plan view of a disposable underpant article of the present invention in a preassembled flat configuration.

FIG. 4 is a perspective view of a full-sized, disposable underpant of the present invention.

FIG. 5 is a perspective view of a full-sized, disposable underpant of the present invention.

FIG. 6 is a sectioned view taken along view lines 6-6 of FIG. 1a and illustrating the outer cover liner topsheet layer and elastics.

FIG. 7 is a sectioned view taken along view lines 6-6 of FIG. 2b and illustrating the outer cover liner topsheet layer and elastics.

FIG. 8 is an exploded sectioned view taken along view lines 4-4 of FIG. 1a and illustrating the absorbent layer, barrier, and outer cover.

FIG. 9 is an exploded sectioned view taken along view lines 4-4 of FIG. 2a and illustrating the absorbent layer, barrier, and outer cover.

FIG. 10 is an expanded plan view of a disposable underpant article of the present invention in a preassembled flat configuration and showing a skewed forward placement of the absorbent pad.

FIG. 11 is an expanded plan view of a disposable underpant article of the present invention in a preassembled flat configuration and showing a skewed forward placement of the absorbent pad.

FIG. 12 is an expanded plan view of a disposable underpant article of the present invention in a preassembled flat configuration and showing a skewed forward placement of a profile absorbent pad.

FIG. 13a is a cross-sectional view of the absorbent pad taken along section line 4-4 of FIG. 1a and showing a homogeneous distribution of fibrous and high absorbency material.

FIG. 13b is a cross-sectional view of the absorbent pad taken along section line 4-4 of FIG. 1b and showing a homogeneous distribution of fibrous and high absorbency material which has a profiled basis weight distribution.

FIG. 14 is a cross-sectional view of the absorbent pad taken along section line A-A of FIG. 1a and showing a homogeneous distribution of fibrous and high absorbency material.

FIG. 15a is a cross-sectional view of the absorbent pad taken along section line 4-4 of FIG. 1a and showing a layered distribution of fibrous and high absorbency material.

FIG. 15b is a cross-sectional view of the absorbent pad taken along section line A-A of FIG. 1a and showing a pulsed distribution of fibrous and high absorbency material with little high absorbency material in the ends.

FIG. 15c is a cross-sectional view of the absorbent pad taken along section line 4-4 of FIG. 1a and showing a non-uniform distribution of fibrous and high absorbency material in the cross direction of the absorbent pad.

### DETAILED DESCRIPTION

The article and method of the present invention provide an absorbent underpant having a three dimensional, disposable, discrete underpant including elasticized leg openings and waist portion circumferentially stretchable about the hip and stomach regions, providing incontinence protection.

The present invention includes an absorbent underpant providing a protection obtained by a flexible absorbent core associated with a crotch area of the underpant wherein the absorbent core is maintained in proper location for incontinence discharge by an elastic system surrounding each leg opening. The absorbent core extends from the crotch area of the underpant into the body of the underpant in front and back body portions. The underpant of the present invention is capable of trapping and absorbing incontinence discharge and preventing liquid strike-through onto outer clothing and bed linen.

The present invention provides an absorbent underpant having preferred absorption, containment, and comfort. The underpant of the present invention provides an absorbent core disposed in the front section and the central section such that the absorbent core is placed asymmetrically in the longitudinal dimension of the garment. The asymmetrical placement of the absorbent pad in the longitudinal dimension of the garment facilitates the formation of an adequate and comfortable garment when transformed from a flat condition to an anatomically-conforming shape.

A proportion skew factor of the absorbent core length in the back section divided by the combined absorbent core length of the central section plus the front section of the underpant is less than about 0.10. In one aspect, the proportion skew factor of the present invention is less than 0.095, preferably less than 0.085, and most preferably less than 0.07. The disposable underpant provides an elasticized article facilitating the formation of a pouch structure in the central section, and an effective seal between the disposable underpant and the wearer. The disposable underpant of the present invention is comfortable to wear and has preferred containment characteristics.

A preferred embodiment of the present invention provides an absorbent core facilitating rapid aqueous liquid transfer in the x, y, and z directions by a continuous and constant proportion of fiber and superabsorbent in the CD and MD directions throughout the dimensions of the absorbent core. In one aspect, the absorbent core has differential densities and zones of fiber or superabsorbent in the CD and MD direction throughout the dimensions of the absorbent core.

In one aspect, the present invention provides a disposable underpant for use in absorbing and containing human body waste including a surge layer of intake material to contain large aqueous liquid gushes between the topsheet layer and the absorbent core.

In one aspect, the present invention provides a disposable underpant for use in absorbing and containing human body waste including a pledget positioned between the absorbent core and the liquid barrier surge layer of intake material to contain large aqueous liquid gushes between the topsheet layer and the absorbent core.

In one embodiment, the present invention provides a disposable underpant including both a pledget and a surge layer of intake material.

In one embodiment, the pledget and surge layer of intake material are skewed into the front and central sections of the disposable underpant and are not present in the back section.

By “aqueous-liquid-impervious” is meant a layer or laminate which will contain aqueous liquid such as urine and will not pass through the layer or laminate under ordinary use conditions in a direction perpendicular to the plane of the layer or laminate at the point of aqueous liquid contact.

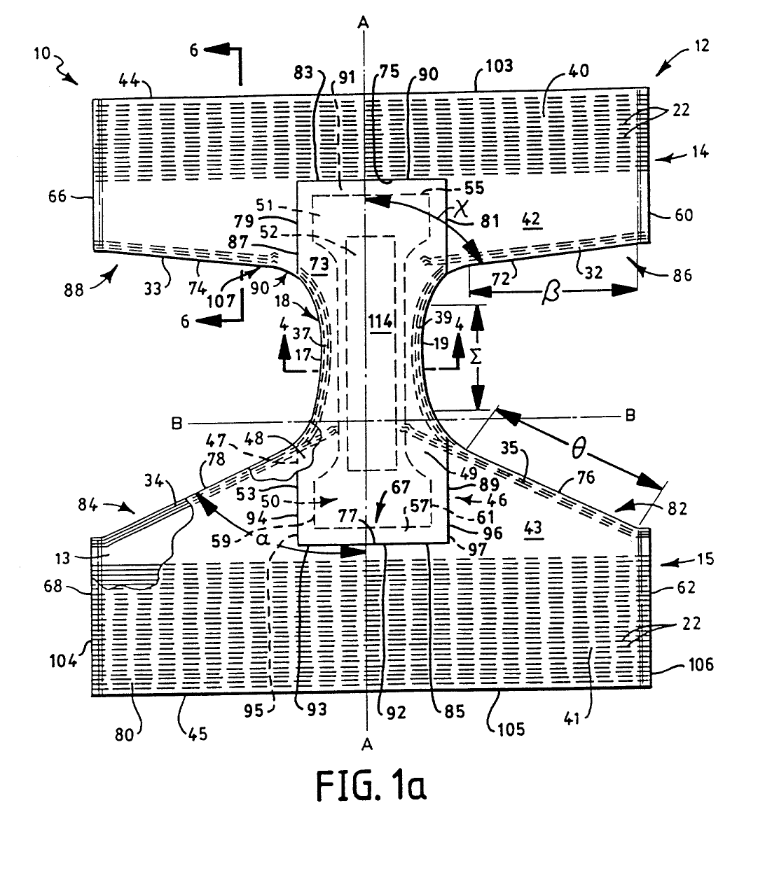

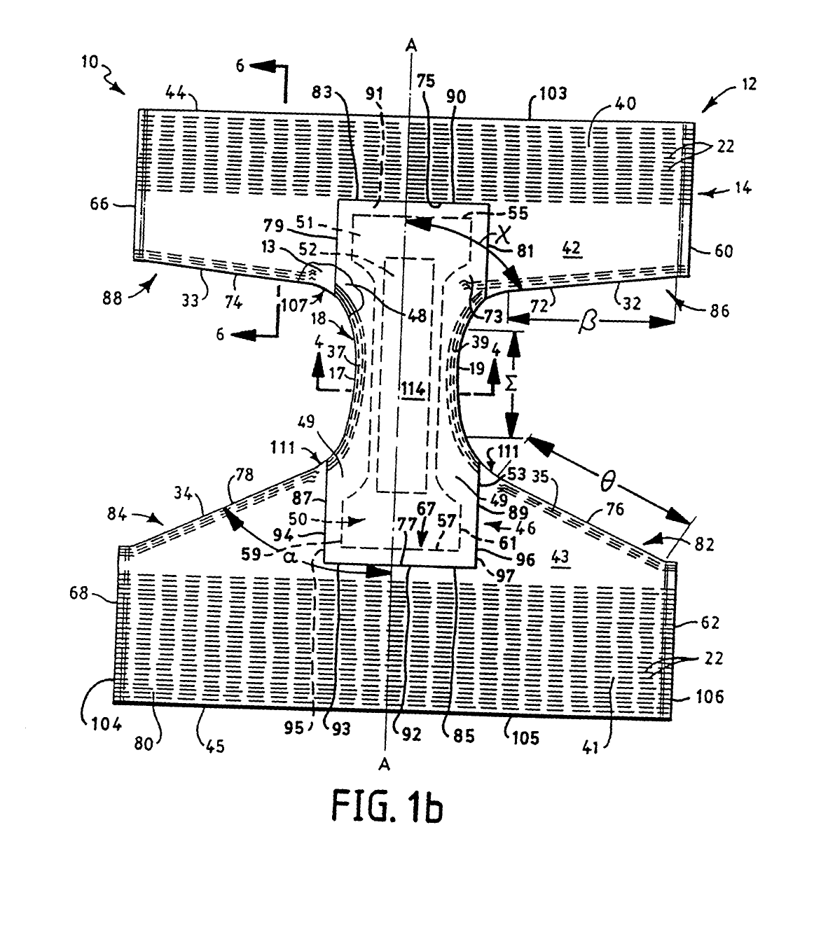

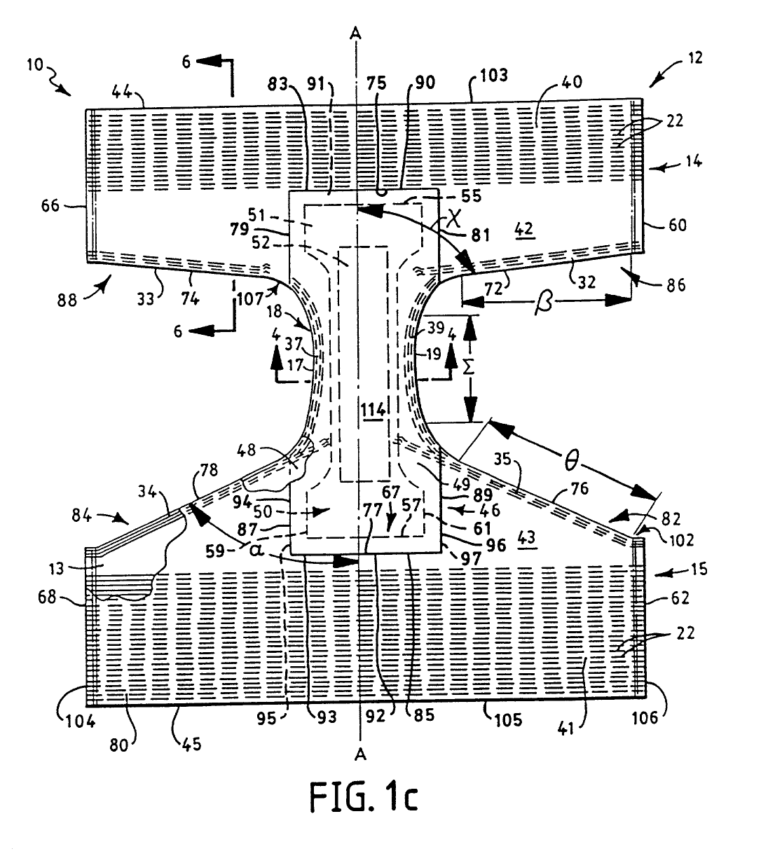

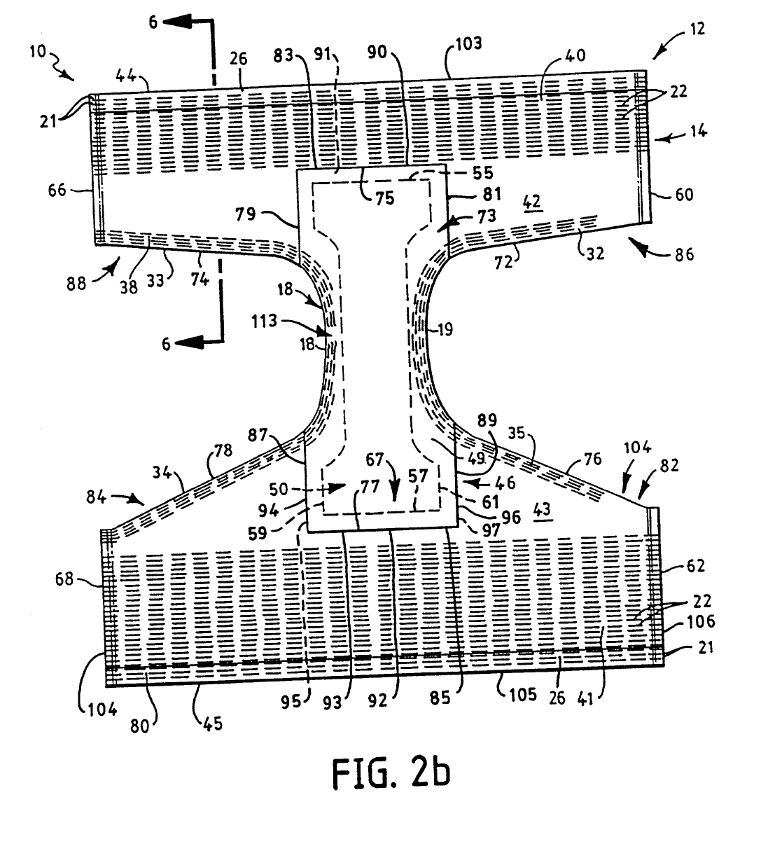

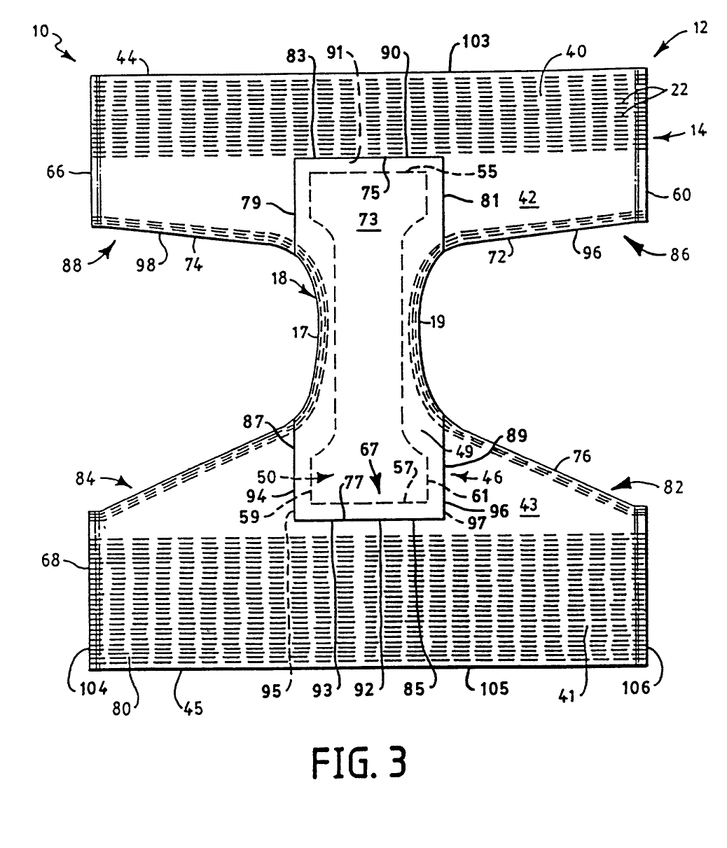

The back, back side, or back portion with reference to the human anatomy are defined by reference to FIG. 1. FIG. 3 illustrates a transverse axis or plane passing through the center of the illustrated underpant to divide it into a front half and a back half. The “back” or “back side” or “back portion” of the wearer will include that portion from the centerline on one side of the wearer and around the back to a similar point on the other side of the wearer.

Back section is the back one third of the total garment length which is worn on the posterior side of the wearer's body.

Barrier fabric or barrier means a fabric which is relatively impervious to the transmission of aqueous liquids, i.e., a fabric which has a blood strike-through rate of 1.0 or less according to ASTM test method 22.

Barrier fabric refers to a fabric having a useful level of resistance to penetration by aqueous liquid and/or particulates. Resistance to aqueous liquid penetration is measured by hydrostatic head tests, strike-through tests, and water spray penetration tests. Unless otherwise specified, a material with resistance to aqueous liquid penetration refers to a material having a hydrostatic head of at least about 20 centimeters as determined in accordance with the standard hydrostatic pressure test AATCCTM No. 127-1977. For example, such a aqueous liquid resistant material may have a hydrostatic head of 60 centimeters or more. Resistance to penetration by particulates is measured by determining the air filter retention of dry particles and can be expressed as a particles holdout efficiency. In particular, particle hold-out efficiency refers to the efficiency of a material at preventing the passage of particles of a certain size range through the material. Particle holdout efficiency is measured by determining the air filter retention of dry particles utilizing tests such as, for example, IBR Test Method No. E-217, Revision G (Jan. 15, 1991) performed by InterBasic Resources, Inc. of Grass Lake, Mich. A high particle holdout efficiency is preferred for barrier fabrics. Preferably, barrier fabrics should resist penetration by a column of tap water of at least about 20 cm and/or should have a particle hold-out efficiency of at least about 40 percent for particles having a diameter greater than about 0.1 micron.

Blend means a mixture of two or more polymers, while the term “alloy” means a sub-class of blends wherein the components are immiscible but have been compatibilized. “Miscibility” and “immiscibility” are defined as blends having negative and positive values, respectively, for the free energy of mixing.

Further, “compatibilization” is defined as the process of modifying the interfacial properties of an immiscible polymer blend in order to make an alloy.

Bonded refers to the joining, adhering, connecting, attaching, or the like, of two elements. Two elements will be considered to be bonded together when they are bonded directly to one another or indirectly to one another, such as when each is directly bonded to intermediate elements.

Bonded carded web refers to webs made from staple fibers sent through a combing or carding unit, which breaks apart and aligns the staple fibers in the machine direction to form a machine direction oriented fibrous nonwoven web. Such fibers usually are purchased in bales placed in a picker which separates the fibers prior to the carding unit. The web is formed and then is bonded by one or more of several known bonding methods. One such bonding method is powder bonding, wherein a powdered adhesive is distributed through the web and then activated, usually by heating the web and adhesive with hot air. Another suitable bonding method is pattern bonding, wherein heated calender rolls or ultrasonic bonding equipment bond the fibers together, usually in a localized bond pattern, though the web can be bonded across its entire surface if so desired. Another suitable bonding method, particularly when using bicomponent staple fibers, is through-air bonding.

Bulk refers to the thickness of samples measured with a Model 49-70 thickness tester available from TMI (Testing Machines Incorporated) of Amityville, N.Y. The thickness tester was equipped with a 2-inch diameter circular foot and measurements were taken at an applied pressure of about 0.2 pounds per square inch (psi). Bulk measurements of samples that are substantially dry, i.e., having a moisture content less than about 10 percent, by weight, as determined by conventional methods, is referred to as dry bulk.

The term “cross machine direction” or CD means the width of fabric, i.e., a direction perpendicular to the “machine direction” or MD.

CD direction is the cross or short direction of the product and is generally perpendicular to the MD or machine direction.

Cellulosic fibers refer to fibers comprising cellulose, a linear, water-wettable polysaccharide, whether existing as a single constituent in a larger natural aggregate such as wood pulp, bagasse and cotton linters, or as a derivative of the natural aggregate such as alpha pulp or viscose rayon.

Central section is the central one third of the total garment length which is between the front and back regions of the product on the wearer's body.

Closely adjacent means one element is positioned as close to another element as can be accomplished because of other nearby structure, manufacturing restraints, comfort, or fit considerations.

Coform means a process in which at least one meltblown diehead is arranged near a chute through which other materials are added to the web while it is forming. Such other materials are pulp, superabsorbent particles, cellulose, or staple fibers. Coform processes are shown in commonly assigned U.S. Pat. Nos. 4,818,464 to Lau and 4,100,324 to Anderson et al. Webs produced by the coform process are referred to as coform materials.

Consisting essentially of does not exclude the presence of additional materials which do not significantly affect the desired characteristics of a given composition or product. Exemplary materials of this sort would include, without limitation, pigments, antioxidants, stabilizers, surfactants, waxes, flow promoters, particulates, and materials added to enhance processability of the composition.

Continuous means that the described structure is a closed-loop structure. The continuous structure is unitary, i.e., a one-piece structure, or is made up of individual elements suitably joined together to form a closed-loop.

Disposable means that the described garment or article is designed to be used until soiled, either by urination, defecation, or otherwise, and then discarded, rather than being washed and reused again. Disposable is not limited to single use or limited use articles but also refers to articles that are so inexpensive to the consumer that they can be discarded if they become soiled or otherwise unusable after only one or a few uses.

Disposed, disposed on, disposed with, disposed at, or disposed near are intended to mean that one element can be integral or unitary with another element, or that one element can be a separate structure joined to or connected to or placed with or placed near another element.

Elastic or elastomeric when referring to a fiber, film, or fabric means a material which upon application of a biasing force, is stretchable to a stretched, biased length at least about 150 percent, or one and a half times, its relaxed, un-stretched length, and which will recover at least 50 percent of its elongation upon release of the stretching, biasing force.

Elasticity, elastic, or elasticized refers to that property of a material or composite elastic material that permits it to recover at least a portion of its original size and shape after removal of the force causing the deformation, expressed in %.

Elasticizable describes a temporarily inhibited elasticized or elastic member which can be activated to recover its elasticity.

Elasticized means that a material is naturally non-elastic is rendered elastic by joining it to an elastic material.

Elongation means the ratio of the extension of a material to the length of the material prior to the extension expressed as a percent, as represented by Equation 1 (Eq. 1).

Extended length−Original length/Original length×100. (Eq. 1)

Extensible, elongatable, stretch, stretchability, or stretch characteristics means that the material can have its length increased, expressed in units of length.

Extension, extend, or extended refers to an increased change in length of a material because of stretching, and is expressed in units of length.

Fabric is used to refer to all of the woven, knitted, and nonwoven webs.

Filament refers to an element having a high ratio of length to diameter or width and includes a fiber, thread, strand, yarn, or combination of these elements.

Finished product means a product that has been manufactured for its intended purpose.

Flexible refers to materials which are compliant and readily conform to the general shape and contours of the human's body.

Front, front side, or front portion include the front part of an article or garment complementary to the “back,” “back side,” or “back portion.”

Front or back are used throughout this description to designate relationships relative to the garment itself, rather than to suggest any position the garment assumes when it is positioned on a wearer.

Front section is the forward one third of the total garment length which is worn on the anterior side of the wearer's body.

Fully gathered with reference to, for example, an opening or border means that the material about the opening or border is gathered along its total periphery.

Garment means any type of non-medically oriented apparel which may be worn, and includes industrial work wear and coveralls, underpants, pants, shirts, jackets, gloves, or socks. Garment means any type of apparel which may be worn, and includes industrial work wear and coveralls, underpants, pants, shirts, jackets, gloves, socks, and the like.

Hydrophilic describes fibers or surfaces of fibers that are wettable by the aqueous liquids in contact with the fibers. The degree of wetting of the materials can be described in terms of contact angles and the surface tensions of the liquids and materials involved. Equipment and techniques suitable for measuring the wettability of particular fiber materials or blends of fiber materials can be provided by a Cahn SFA-222 Surface Force Analyzer System. When measured with the Cahn system, fibers having contact angles less than 90° are designated “wettable,” i.e., “hydrophilic,” and fibers having contact angles greater than 90° are “nonwettable,” i.e., “hydrophobic.”

Intake layer, intake material, or surge layer refers to a material designed to decelerate and diffuse surges of aqueous liquid introduced to the absorbent pad. Examples of these materials are described in U.S. Pat. Nos. 5,192,606 issued Mar. 9, 1993, to D. Proxmire et al.; 5,486,166 issued Jan. 23, 1996 to Ellis et al.; 5,490,846 issued Feb. 13, 1996 to Ellis et al.; and 5,509,915 issued Apr. 23, 1996 to Hanson et al.; the disclosures of which are hereby incorporated by reference.

Integral is used to refer to various portions of a single unitary element rather than separate structures bonded to or placed with or placed near one another.

Inward or outward refers to positions relative to the center of an absorbent garment, and particularly transversely and/or longitudinally closer to or away from the longitudinal and transverse center of the absorbent garment.

Joining, join, joined, or variations thereof, when used in describing the relationship between two or more elements, means that the elements can be connected together in any suitable manner, such as by heat sealing, ultrasonic bonding, thermal bonding, adhesives, stitching, or the like. Further, the elements can be joined directly together, or may have one or more elements interposed between them, all of which are connected together.

Layer when used in the singular can have the dual meaning of a single element or a plurality of elements.

Liquid means a substance and/or material that flows and will assume the interior shape of a container into which it is poured or placed. For this specification, aqueous liquid means an aqueous fluid material.

Liquid communication or liquid migration refers to the ability of an aqueous liquid to travel through or between or along two structures in the absence of an aqueous liquid impervious barrier preventing aqueous liquid travel between or along the two structures.

Liquid impervious when used in describing a layer or laminate including at least one aqueous liquid impervious film or layer and at least one aqueous liquid pervious film or layer means that the aqueous liquid will not pass through the laminate, under ordinary use conditions, in a direction generally perpendicular to the plane of the laminate at the point of aqueous liquid contact. Liquid may spread or be transported parallel to the plane of the aqueous liquid impervious film or layer, but is not considered to be within the meaning of “aqueous liquid impervious” when used with reference to the laminate.

Machine direction or MD means the length of a fabric in the direction in which it is produced. The term “cross machine direction” or CD means the width of fabric, i.e. a direction generally perpendicular to the MD.

MD direction is the longitudinal or long direction of the product and is typically the direction in which the product is manufactured.

Member when used in the singular can have the dual meaning of a single element or a plurality of elements.

Microfibers mean small diameter fibers having an average diameter not greater than about 75 microns, for example, having an average diameter of from about 0.5 microns to about 50 microns, or more particularly, microfibers may have an average diameter of from about 2 microns to about 40 microns. Another frequently used expression of fiber diameter is denier, which is defined as grams per 9000 meters of a fiber and is calculated as fiber diameter in microns squared, multiplied by the density in grams/cc, multiplied by 0.00707. A lower denier indicates a finer fiber and a higher denier indicates a thicker or heavier fiber. For example, the diameter of a polypropylene fiber given as 15 microns is converted to denier by squaring, multiplying the result by 0.89 g/cc and multiplying by 0.00707. Thus, a 15 micron polypropylene fiber has a denier of about 1.42 (15²×0.89×0.00707=1.415). Outside the United States the unit of measurement is more commonly the “tex,” which is defined as the grams per kilometer of fiber. Tex is calculated as denier/9.

Monocomponent fiber refers to a fiber formed from one or more extruders using only one polymer, and is not meant to exclude fibers formed from one polymer to which small amounts of additives have been added for coloration, anti-static properties, lubrication, or hydrophilicity. These additives, e.g. titanium dioxide for coloration, are generally present in an amount less than 5 weight percent and more typically about 2 weight percent.

Multilayer laminate means a laminate wherein some of the layers are spunbond and some are meltblown such as a spunbond/-meltblown/spunbond (SMS) laminate and others as disclosed in U.S. Pat. Nos. 4,041,203 to Brock et al., 5,169,706 to Collier, et al, 5,145,727 to Potts et al., 5,178,931 to Perkins et al. and 5,188,885 to Timmons et al. Such a laminate is made by sequentially depositing onto a moving forming belt first a spunbond fabric layer, then a meltblown fabric layer and last another spunbond layer and then bonding the laminate in a manner described below. Alternatively, the fabric layers are made individually, collected in rolls, and combined in a separate bonding step. Such fabrics usually have a basis weight of from about 0.1 to 12 osy (6 to 400 gsm), or more particularly from about 0.75 to about 3 osy.

Multilayer laminates may also have various numbers of meltblown layers or multiple spunbond layers in many different configurations and may include other materials like films (F) or coform materials, e.g., SMMS, SM, SFS, etc.

Non-elastic refers to any material that does not fall within the definition of “elastic.”

Nonwoven fabric or nonwoven web means a web having a structure of individual fibers or threads which are interlaid, but not in an identifiable manner as in a knitted fabric. Nonwoven fabrics or webs have been formed from many processes such as for example, meltblowing processes, spunbonding processes, and bonded carded web processes. The basis weight of nonwoven fabrics is expressed in ounces of material per square yard (osy) or grams per square meter (gsm) and the fiber diameters are expressed in microns. To convert from osy to gsm, multiply osy by 33.91.

Nonwoven web means a web of material formed without the aid of a textile weaving or knitting process, or a web having a structure of individual fibers or threads that are interlaid, but not in any identifiable, repeating pattern. Nonwoven webs have been formed by a variety of processes such as, for example, meltblowing processes, spunbonding processes, and bonded carded web processes. Nonwoven web means a web of material formed without the aid of a textile weaving or knitting process. Nonwoven webs have been formed from many processes such as, for example, meltblowing processes, spunbonding processes, and bonded carded web processes. The basis weight or nonwoven fabrics is expressed in ounces of material per square yard (osy) or grams per square meter (gsm) and the fiber diameters are expressed in microns.

Operatively joined, elastically associated, or associated with reference to the attachment of an elastic member to another element means that the elastic member when attached to or placed with or formed from the element gives that element elastic properties. With reference to the attachment of a non-elastic member to another element, it means that the member and element can be attached or placed together in any suitable manner that allows or permits them to perform their intended or described function, while not completely inhibiting the properties of the individual elements. The attaching or placing can be either directly, such as attaching or placing either member directly with an element, or can be indirectly by means of another member or element disposed between the first member and the first element. In the joining of an elastic member to a non-elastic member, the two joined members can exhibit elasticity or elastic properties. Operatively joined, with reference to the attachment of an elastic member to another element, means that the elastic member when attached to or connected to the element, or treated with heat or chemicals, by stretching, or the like, gives the element elastic properties; and with reference to the attachment of a non-elastic member to another element, means that the member and element can be attached in any suitable manner that permits or allows them to perform the intended or described function of the joinder. The joining, attaching, connecting or the like can be either directly, such as joining either member directly to an element, or can be indirectly by means of another member disposed between the first member and the first element.

Outward refers to a position relative to the center of an absorbent garment, and particularly transversely and/or longitudinally away from the longitudinal and transverse center of the absorbent.

Partially elastic refers to a substrate, garment, a part of a garment, or the like, having at least one portion thereof that is elastic.

Particles as in SAP or SAM means any geometric or non-geometric form such as, but not limited to, spherical grains, cylindrical fibers or strands, flat surfaces or roughened surfaces, sheets, ribbons, strings, strands, or the like. When used in an absorbent structure, the particles can be loosely formed into a shaped structure or compressed into a shaped form.

Permeable or permeability or pervious refers to the ability of a aqueous liquid, such as, for example, a gas to pass through a particular porous material. Permeability is expressed in units of volume per unit time per unit area, for example, cubic feet per minute per square foot of material, e.g., ft³/minute/ft². Permeability was determined utilizing a Frazier Air Permeability Tester available from the Frazier Precision Instrument Company and measured in accordance with Federal Test Method 5450, Standard No. 191A, except that the sample size was 8″×8″ instead of 7″×7″. Although permeability is expressed as the ability of air or other gas to pass through a permeable sheet, sufficient levels of gas permeability may correspond to levels of aqueous liquid permeability to enable the practice of the present invention. For example, a sufficient level of gas permeability may allow an adequate level of aqueous liquid to pass through a permeable sheet with or without assistance of a driving force such as, for example, an applied vacuum or applied gas pressure.

Personal care product means diapers, training pants, absorbent underpants, adult incontinence products, and feminine hygiene products

Polymer includes but is not limited to, homopolymers, copolymers, such as for example, block, graft, random and alternating copolymers, terpolymers, and blends and modifications thereof. Unless otherwise specifically limited, the term “polymer” includes all geometrical configurations of the molecule. Configurations include, but are not limited to isotactic, syndiotactic, and random symmetries.

Pulp refers to pulp containing fibers from natural sources such as woody and non-woody plants. Woody plants include, for example, deciduous and coniferous trees. Non-woody plants include, for example, cotton, flax, esparto grass, milkweed, straw, jute hemp, and bagasse.

Releasably attached, releasably bonded, releasably engaged or variations thereof refer to two elements being connected or connectable such that the elements tend to remain connected absent a separation force applied to one or both of the elements, and the elements being capable of separation without substantial permanent deformation or rupture. The required separation force is beyond that encountered while wearing the absorbent garment.

Retraction refers to a decreasing change in length of an extended material upon removal of the force causing the extension.

Side refers to a position in which a side of the body faces the supporting surface.

Spunbonded fibers refers to small diameter fibers which are formed by extruding molten thermoplastic material as filaments from a plurality of fine, usually circular capillaries of a spinneret with the diameter of the extruded filaments then being rapidly reduced as-by, for example, in U.S. Pat. Nos. 4,340,563 to Appel et al., and 3,692,618 to Dorschner et al., 3,802,817 to Matsuki et al., 3,338,992 and 3,341,394 to Kinney, 3,502,763 to Hartman, and 3,542,615 to Dobo et al. Spunbond fibers are generally not tacky when they are deposited onto a collecting surface. Spunbond fibers are generally continuous and have average diameters (from a sample of at least 10) larger than 7 microns, more particularly, between about 10 and 20 microns.

Staple fiber refers to a natural fiber or a length cut from, for example, a manufactured filament. Staple fibers typically have a length between about 3 and about 7.5 millimeters.

Stretch, stretchability, or stretch characteristics mean that the material can have its length increased, expressed in units of length. See also Extensible.

Stretch bonding refers to a process wherein an elastic member is bonded to another member while only the elastic member is extended at least about 25 percent of its relaxed length. “Stretch bonded laminate” refers to a composite elastic material made according to the stretch bonding process, i.e., the layers are joined together when only the elastic layer is in an extended condition so that upon relaxing the layers, the nonelastic layer is gathered. Such laminates usually have machine directional stretch properties and are stretched to the extent that the nonelastic material gathered between the bond locations allows the elastic material to elongate. One type of stretch bonded laminate is disclosed, for example, by U.S. Pat. No. 4,720,415 to Vander Wielen et al., in which multiple layers of the same polymer produced from multiple banks of extruders are used. Other composite elastic materials are disclosed in U.S. Pat. Nos. 4,789,699 to Kieffer et al., 4,781,966 to Taylor and 4,657,802 and 4,652,487 to Morman and 4,655,760 to Morman et al.

Substrates, surface, or sheet means a layer that is a film or woven web or nonwoven web, a laminate, pervious or impervious to air, gas, and/or aqueous liquids; or a composite structure comprising for example a topsheet, backsheet, and an absorbent medium between the topsheet and backsheet.

Superabsorbent refers to absorbent materials capable of absorbing at least 10 grams of aqueous liquid, e.g., distilled water per gram of absorbent material while immersed in the liquid for 4 hours and holding substantially all of the absorbed aqueous liquid while under a compression force of up to about 1.5 psi.

Surface includes any layer, film, woven, nonwoven, laminate, composite, or the like, whether pervious or impervious to air, gas, and/or aqueous liquids.

Surge layer refers to a material designed to help decelerate and diffuse surges of aqueous liquid that are introduced to the absorbent pad. Examples of surge materials are described in U.S. Pat. Nos. 5,192,606 issued Mar. 9, 1993, to D. Proxmire et al.; 5,486,166 issued Jan. 23, 1996 to Ellis et al.; 5,490,846 issued Feb. 13, 1996 to Ellis et al.; and 5,509,915 issued Apr. 23, 1996 to Hanson et al.; the disclosures of which are hereby incorporated by reference.

Tension refers to a force tending to cause the extension of a body, or to the balancing force within that body resisting the extension. Tension is expressed in units of grams.

Thermoplastic means a material that softens when exposed to heat and which substantially returns to a nonsoftened condition when cooled to room temperature.

Through-air bonding or TAB means a process of bonding a nonwoven bicomponent fiber web in which air, sufficiently hot to melt one of the polymers of which the fibers of the web is forced through the web. The air velocity is between 100 and 500 feet per minute, and the dwell time is as long as 6 seconds. The melting and resolidification of the polymer provides the bonding. Through air bonding has relatively restricted variability. Through-air bonding TAB requires the melting of at least one component to accomplish bonding, and is restricted to webs with two components like conjugate fibers or those which include an adhesive. In the through-air bonder, air having a temperature above the melting temperature of one component and below the melting temperature of another component is directed from a surrounding hood, through the web, and into a perforated roller supporting the web. Alternatively, the through-air bonder is a flat arrangement wherein the air is directed vertically downward onto the web. The operating conditions of the two configurations are similar, the primary difference being the geometry of the web during bonding. The hot air melts the lower melting polymer component and thereby forms bonds between the filaments to integrate the web.

Two-dimensional refers to a garment, such as a diaper, that can be opened and laid in a flat condition without destructively tearing any structure. A two-dimensional garment does not have continuous leg and waist openings when opened and laid flat and requires a refastening device, such as adhesive tapes, to attach about the wearer.

Undergarment refers to a substantially rectangular adult incontinence absorbent product suspended from the wearer by straps attached to the waist regions of the product. Examples are commercially available DEPEND® Elastic Leg Underpants products.

Waistband refers to a border about the waist opening of an underpant and is constructed of one or more layers of material.

Referring now to FIG. 1a, a disposable underpant 12 is shown to illustrate a preferred embodiment of the article of the present invention in a flat configuration prior to assembly.

The following detailed description is made in the context of an article 10 including a disposable underpant 12 having an absorbent core in place during use. The present invention is employed for incontinence, vaginal discharges, perspiration discharges, and the like, in adult, child, and infant products.

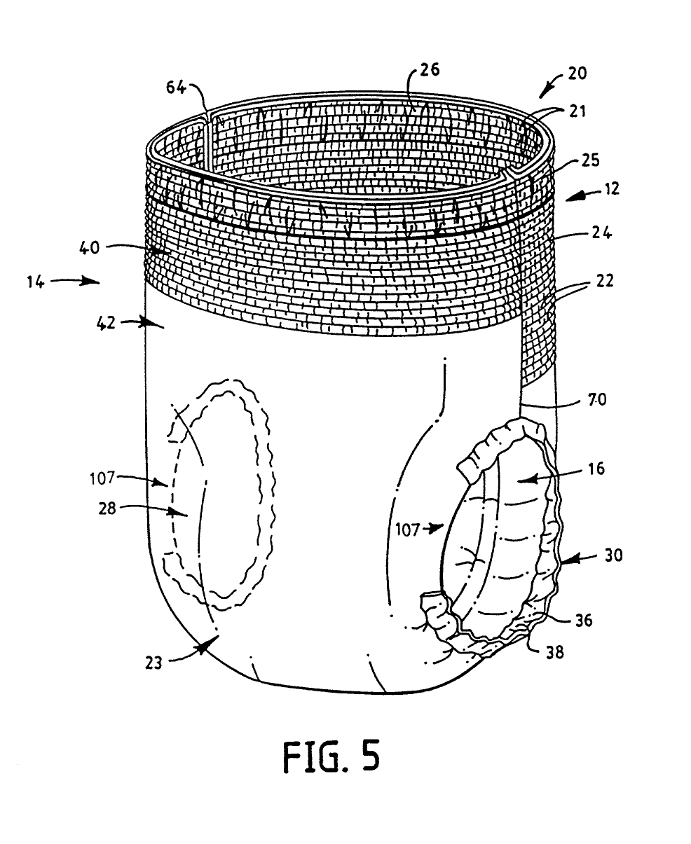

In FIG. 1a, the underpant 12 is shown having an outer cover 13 which includes a front body portion 14, a back body portion 15, a front waist edge portion 44, a back waist edge portion 45, a crotch portion 18, waist liner 26 optionally shown in FIGS. 5 and 7, leg liner 38 optionally shown in FIGS. 5 and 7, and body liner 80. The outer cover 13 includes a front body portion 14, a back body portion 15, a front waist edge portion 44, a back waist edge portion 45, and a crotch portion 18 wherein the crotch portion 18 is interposed between the front and back body portions 14 and 15.

The outer cover 13 is compliant and soft feeling to the wearer. The outer cover 13 is a soft, flexible, porous sheet which is liquid pervious, permitting liquids to penetrate readily into its thickness, or impervious, resistant to the penetration of liquids into its thickness. A suitable outer cover 13 is manufactured from a wide range of materials, such as natural fibers, e.g., wood or cotton fibers, synthetic fibers, e.g., 10 polyester or polypropylene fibers, or from a combination of natural and synthetic fibers or reticulated foams and apertured plastic films.

In one aspect, the outer cover 13 is a woven or nonwoven web or sheet such as a spunbond, meltblown, or bonded-carded web composed of synthetic polymer filaments, such as polypropylene, polyethylene, polyesters, or the like, or a web of natural polymer filaments such as rayon or cotton. In one aspect, the bonded-carded web is thermally bonded or sprayed with a binder. Suitably, the outer cover 13 is a nonwoven spunbond. Preferably, the outer cover 13 is a spunbond polypropylene nonwoven with a wire-weave bond pattern. The spunbond material is available from Kimberly-Clark Corporation, located in Roswell, Ga. The outer cover 13 has a weight in the range from about 0.3 oz. per square yard (osy) to about 2.0 osy and preferably about 0.6 osy. The outer cover 13 of the underpant maybe printed, colored, or decoratively embossed. The outer cover 13 has a pore size which readily allows the passage of air, sweat, or perspiration because of the breathability of the material. In one aspect, the outer cover 13 is selectively embossed or perforated with discrete slits or holes extending through the cover 13.

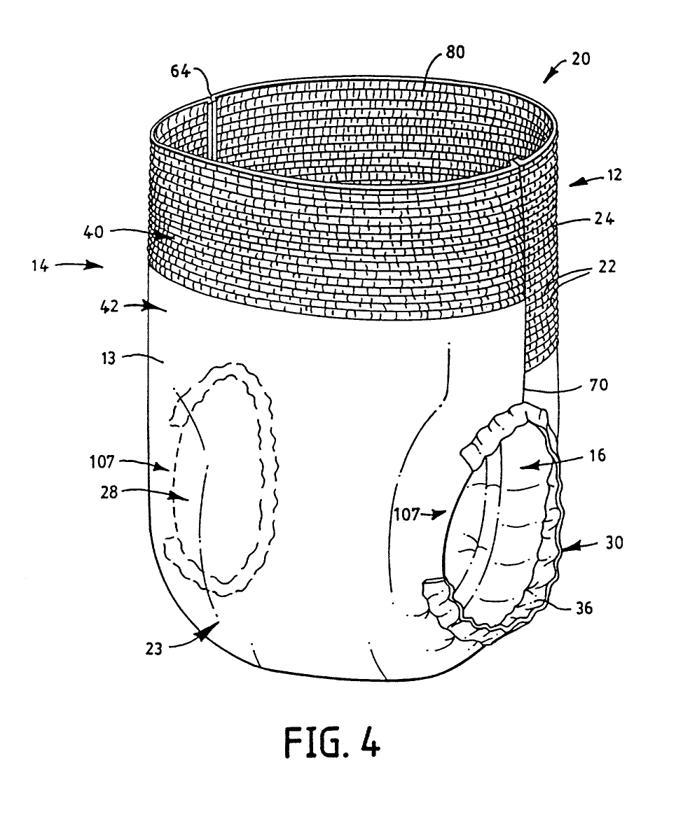

Referring now to FIGS. 1a, 2a, and 3, an edge 60 of front body portion 14 is assembled with an edge 62 of the back body portion 15 to form a seal or side seam 64. An edge 66 of the front body portion 14 is assembled with an edge 68 of the back body portion 15 to form a seal or side seam 70. The assembled front body portion 14 and the back body portion 15 form a waist opening 20 for putting on and taking off the underpant 12. The waist opening 20 is surrounded at least in part by waist portion elastic 22 including from between 1 and 40 elastic strands, threads, ribbons, or bands of elastic material. The waist portion elastic 22 is stretched and attached to the body portions 14 and 15. In one aspect, the waist portion elastic 22 is placed in the front and back waist edge portions 44 and 45. In another embodiment, the waist portion elastic 22 is contained only in the front body portion 14 or only in the back body portion 15. The waist portion elastic 22 is released after attachment to produce waist portion folds, gathers, or pleats 24 (shown in FIGS. 4 and 5) to allow expansion of the waist opening 20 and the body portions 14 and 15 so that the underpant 12 can fit various sized individuals.

Because users prefer a brief style underpant, the front waist edge portion 44 of the underpant 12 preferably comes to the navel and even around the wearer's waist. Having the underpant 12 at the brief style height and then drawing in either or both front and back body portions 14 and 15 with the waist portion elastic 22 provides a snug fit. Alternative underpant styles include bikini, e.g., regular leg cut and french leg cut, and hipster, e.g., regular leg cut or french leg cut.

In one embodiment, the waist opening 20 is surrounded by waist elastic 21 including between 1 and 10 strands, ribbons, or bands of elastic materials stretched and attached to the front waist edge portion 44 and/or the back waist edge portion 45. (See FIGS. 5 and 7.) In another embodiment, the waist elastic 21 is contained only in the front waist edge portion 44 or in the back waist edge portion 45. The waist elastic 21 is released after attachment to produce waist folds, gathers, or pleats 25 to allow expansion of the waist opening 20 so that the underpant 12 can fit various sized individuals. Using waist elastic 21 having a different tension or elongation than the tension or elongation used in the waist portion elastic 22 provides a better fitting underpant 12.

Referring again to FIG. 1a, the front body portion 14 and the back body portion 15 together with the crotch portion 18 forms leg openings 28 and 30, respectively, which are circular or oval in shape. The leg openings 28 and 30 are each surrounded at least in part by leg elastics 32, 33, 34, and 35, respectively. The front leg elastics 32 and 33 are stretched and attached to the front body portion 14. The back leg elastics 34 and 35 are stretched and attached to the back body portion 15. The crotch elastics 37 and 39 are stretched and attached to the crotch portion 18. The leg and crotch elastics 32, 33, 34, 35, 37, and 39 are released after attachment to produce leg gathers, also referred to as folds or pleats 36 to allow expansion of the leg openings 28 and 30 to fit various sized legs.

The front body portion 14 is divided into a front upper body portion 40 and a front lower body portion 42. (See FIGS. 1a, 2a, and 3.) The back body portion 15 is divided into a back upper body portion 41 and a back lower body portion 43. The front and back upper body portions 40 and 41 preferably include waist portion elastic 22 capable of stretching to allow the wearer to put on the underpant 12 and then readily contracting and conforming to the wearer's body. The waist portion elastic 22 provides a close or snug fit to different body shapes and sizes. The waist portion elastic 22 is positioned on both the front and the back upper body portions 40 and 41, respectively, at positions between the waist opening 20 and the leg openings 28 and 30, so that the underpant 12 fits the wearer better, particularly around the body. The front and back lower body portions 42 and 43 do not necessarily require waist portion elastic 22. The basis weight of the laminate forming the outer cover 13, the body liner 80, and waist portion elastic 22 is as high as 5 osy.

The crotch portion 18 of the underpant 12 includes an absorbent barrier composite 46. The absorbent barrier composite 46 extends into the front body portion 14 and/or the back body portion 15. (See FIG. 1a.) The absorbent barrier composite 46 further includes a liquid barrier 48, a substantially liquid-pervious topsheet layer 49, and an absorbent core 50 sandwiched between the liquid barrier 48 and the topsheet layer 49. The liquid barrier 48 and the topsheet layer 49 are longer and wider than the absorbent core 50, so that the peripheries of the liquid barrier 48 and the topsheet layer 49 form margins sealed together using ultrasonic bonds, thermal bonds, or adhesives. In the sealed area, the crotch elastics 37 and 39 are incorporated between the liquid barrier 48 and the topsheet layer 49. The absorbent core 50 is attached to the liquid barrier 48 and/or the topsheet layer 49 using ultrasonic bonds or adhesives. (See FIGS. 1a, 8, and 9.)

The absorbent barrier composite 46 includes additional components to assist in the acquisition, distribution, and storage of body exudates. For example, the absorbent barrier composite 46 includes a transport layer, such as described in U.S. Pat. No. 4,798,603 issued Jan. 17, 1989, to Meyer et al., or a surge management layer, such as described in European Patent Application EP 0 539 703 A1, published May 5, 1993, which patent and application are incorporated herein by reference. Such layers are referred to as acquisition/distribution layers.

The absorbent barrier composite 46 is constructed by supplying topsheet layer 49 and liquid barrier 48 materials and sandwiching an individual absorbent core 50 between the liquid barrier 48 and the topsheet layer 49. The side and end peripheries of the liquid barrier 48 and the topsheet layer 49 outward of the absorbent core 50 are joined with the crotch portion 18, the front body portion 14, and the back body portion 15 and sealed together. The absorbent barrier composite 46 and/or the absorbent core 50 optionally is T-shaped, I-shaped, oval-shaped, hourglass-shaped, rectangular-shaped, or irregularly-shaped. The absorbent barrier composite 46 and/or absorbent core 50 include leg cutouts 29 and 31 opposing indentations in the longitudinal sides 87 and 89 of the absorbent barrier composite 46, the longitudinal side edges 59 and 61 of the absorbent core 50, and/or the longitudinal side edges 104 and 106 of the disposable underpant 12. Leg cutouts 29 and 31 provide a preferred fit of the disposable underpant 12 as the reduced bulk between the wearer's legs reduces or prevents gapping, thereby preventing leaks as well as improving comfort. The other materials used in the disposable underpant 12, including but not limited to the topsheet layer 49, the liquid barrier 48, and outer cover 13 are shaped to include leg cutouts 29 and 31. In one embodiment, the absorbent core 50 is shaped to include leg cutouts 29 and 31, and the topsheet layer 49, the liquid barrier 48, and the outer cover 13 are shaped not to include leg cutouts 29 and 31. In the absorbent barrier composite 46 and/or absorbent core 50, leg cutouts 29 and 31 are not placed symmetrically in disposable underpant 12 but are skewed toward the front end edge 103 of the disposable underpant 12.

In one aspect, geometric shapes are rectangular, oval or racetrack patterns, hourglass configurations, bilobal shapes, and any shape where the length is greater or less than the width.

Referring now to FIGS. 1a, 1b, 1c, 2a, 2b, 3, 10, 11, and 12, the absorbent core 50 is a rectangular shape and includes a peripheral edge 67 having side edges 59 and 61, a front end edge 55, and a back end edge 57. The absorbent core 50 has an exterior surface 63 facing away from the wearer and an interior surface 65 facing toward the wearer.

The porous fibrous matrix of absorbent core 50 is preferably an air laid batt of fluff and high absorbency material formed, for example, according to the teaching of Mazurak and Fries as set forth in U.S. Pat. No. 4,381,782, the entire disclosure of which is incorporated herein by reference.

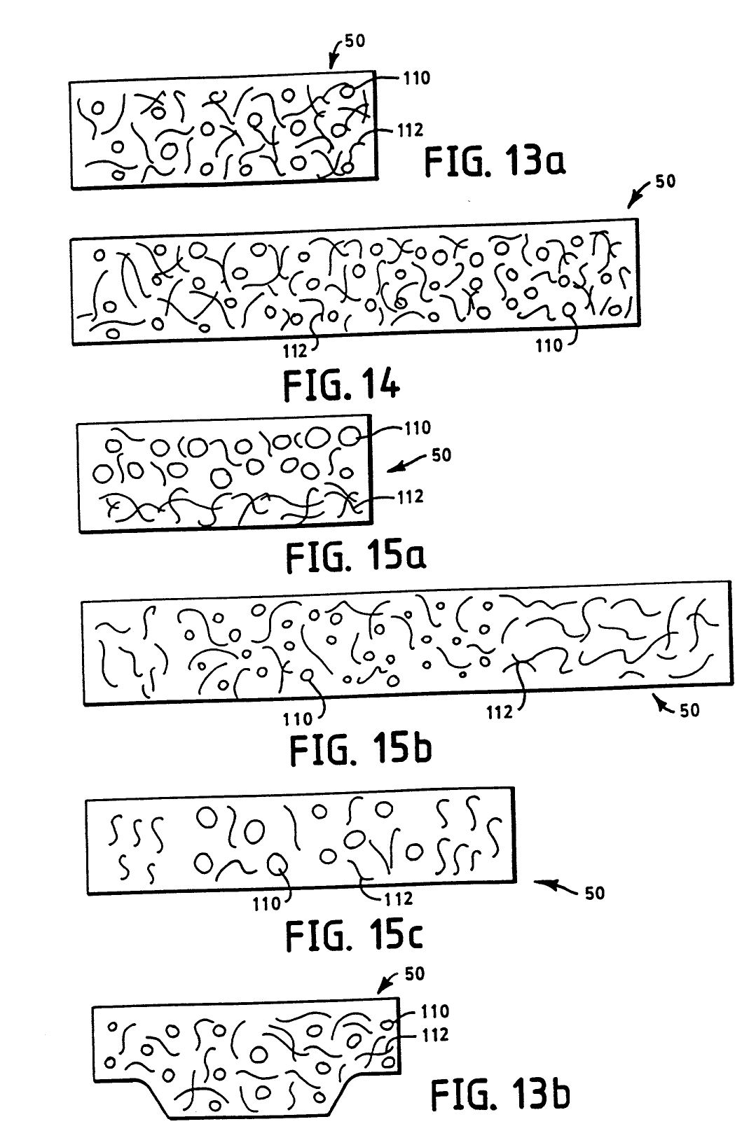

Referring now to FIGS. 13a, 13b, 14, 15a, 15b, and 15c, the absorbent core 50 includes an air-formed mixture of high absorbency material (SAP) 110 and fibers 112, preferably of fluff pulp. Most preferably, as shown in FIGS. 13a, 13b, and 14, the mixing of the fluff fibers 112 and the high absorbency material 110 is homogeneous. In other embodiments, as shown in FIGS. 15a, 15b, and 15c, the mixtures are layered (FIG. 15a), phased to place the high absorbency material in a specific machine direction location (FIG. 15b), or placed in a narrow band in the cross direction (FIG. 15c). In one aspect, fibers other than fluff pulp, such as chemically stiffened and thermo-mechanical pulps, are used. In one aspect, the absorbent core 50 includes absorbent material other than air formed fluff and SAP. For example, coform materials as referenced in U.S. Pat. Nos. 4,818,464 to Lau and 4,100,324 to Anderson are used to make the absorbent as long as they also contain high absorbency materials. In one aspect, wet formed composite materials are used including a combination of fibers and high absorbency materials as disclosed in U.S. Pat. No. 5,651,862 to Anderson et. al. In one aspect, stabilized airlaid materials are used as absorbent materials including mixtures of fibers, binder fibers, and high absorbency materials bound together by latex binding or through air bonding.

The hydrogel polymers are sufficiently cross-linked to render the materials substantially water-insoluble. Cross-linking is by irradiation or by covalent, ionic, van der Waals, or hydrogen bonding. Suitable materials are available from Dow Chemical Company (Drytech 2035 LD), Hoechst-Celanese Corporation, and Allied-Colloid. The high-absorbency material is capable of absorbing at least about 15 times its weight in water, and preferably is capable of absorbing more than about 25 times its weight in water.

The high-absorbency material is distributed or otherwise incorporated into the absorbent core 50. As illustrated in FIGS. 13a, 13b, 14, 15a, 15b, and 15c, the high-absorbency material is substantially uniformly distributed among the fibers of the absorbent core 50. In one aspect, the materials are non-uniformly distributed within the absorbent core 50 fibers to form a continuous gradient of either an increasing or decreasing concentration of high-absorbency material, as determined by observing the concentration moving inward from the liquid barrier 48. In one aspect, the high-absorbency material includes a discrete layer separate from the fibrous material of the absorbent core 50, or a discrete layer integral with the absorbent core 50.

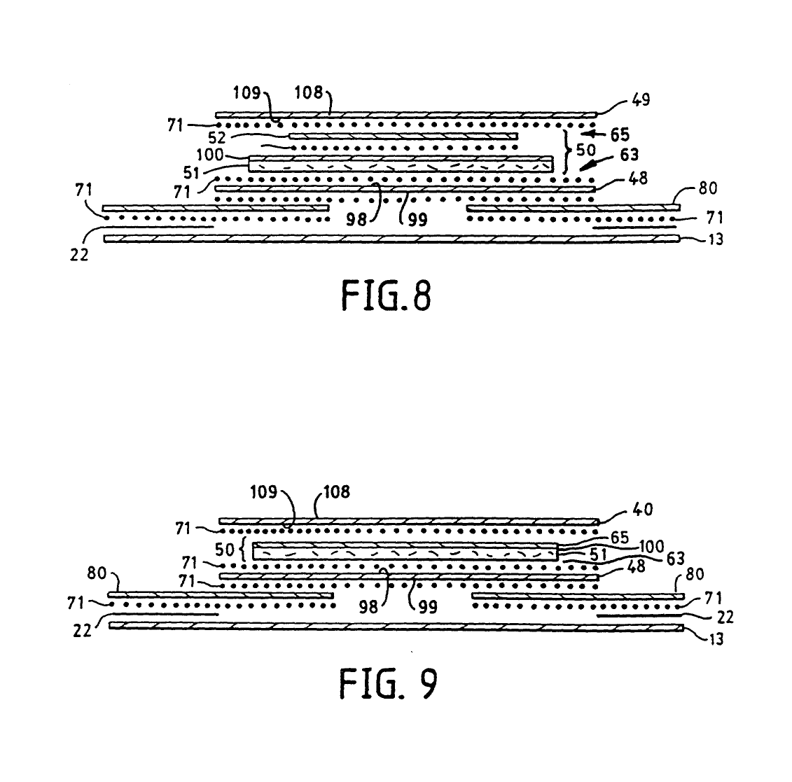

In one aspect, the absorbent pad 50 includes a wrap layer 100 to maintain the integrity of the fibrous core. (See FIG. 8.) The wrap layer 100 includes a cellulosic tissue or spunbond, meltblown, or bonded-carded web material composed of synthetic polymer filaments, such as polypropylene, polyethylene, polyesters, or natural polymer filaments such as rayon or cotton.

The absorbent core 50 preferably has an aqueous liquid capacity great enough to absorb discharges from about 10 grams to about 1500 grams. The absorbent core 50 preferably has a capacity and a thickness preferably less than about 25 mm, thus providing a non-bulky and flexible fit. The capacity of the absorbent core 50 has a total capacity of about 200 grams to about 1300 grams. Preferably, the absorbent core 50 has a total capacity of at least about 300 grams and not more than about 1200 grams. More preferably, the total capacity of the absorbent core 50 is from about 400 grams to about 800 grams.

The total capacity of the absorbent core 50 is determined using the absorbent barrier composite 46 of the underpant 12 and the outer cover 13. The saturated retention capacity is a measure of the total absorbent capacity of an absorbent article 10, in this case, a disposable underpant 12. The saturated retention capacity is determined as follows. The disposable underpant 12 to be tested, having a moisture content of less than about 7 weight percent, is weighed and submerged in an excess quantity of the room temperature (about 23° C.) saline solution described below. The material is allowed to remain submerged for 20 minutes. After 20 minutes, the disposable underpant 12 is removed from the saline solution and placed on a Teflon™ coated fiberglass screen having 0.25 inch openings (commercially available from Taconic Plastics Inc., Petersburg, N.Y.) which, in turn, is placed on a vacuum box and covered with a flexible rubber dam material. A vacuum of 3.5 kilopascals (0.5 pounds per square inch) is drawn in the vacuum box for a period of 5 minutes. The disposable underpant 12 is weighed. The amount of aqueous liquid retained by the material being tested is determined by subtracting the dry weight of the disposable underpant 12 from the wet weight of the disposable underpant 12 after application of the vacuum and is reported as the saturated retention capacity in grams of aqueous liquid retained.

The saline solution is an aqueous solution of about 0.9 percent sodium chloride by weight. A suitable product is S/P™ Certified Blood Saline commercially available from Baxter Diagnostics in McGaw Park, Ill.

The absorbent core 50 includes materials adapted to absorb and retain urine, menses, blood, or other body excrement. The absorbent core 50 includes various natural or synthetic absorbent materials, such as cellulose fibers, surfactant treated meltblown fibers, wood pulp fibers, regenerated cellulose or cotton fibers, or a blend of pulp and other fiber. One such material is a coform material composed of a mixture of cellulosic fibers and synthetic polymer fibers. In one aspect, the absorbent core 50 includes compounds to increase its absorbency, such as 0-95 weight percent of organic or inorganic high-absorbency materials capable of absorbing at least about 15 and up to more than 25 times their weight in water.

Organic high-absorbency materials include natural materials, such as pectin, guar gum and peat moss, and synthetic materials, such as synthetic hydrogel polymers. Such hydrogel polymers include, for example, carboxymethylcellulose, alkali metal salts of polyacrylic acids, polyacrylamides, polyvinyl alcohol, ethylene maleic anhydride copolymers, polyvinyl ethers, hydroxypropyl cellulose, polyvinyl morpholinone, polymers and copolymers of vinyl sulfonic acid, polyacrylates, polyacrylamides, or polyvinyl pyridine. Other suitable polymers include hydrolyzed acrylonitrile grafted starch, acrylic acid grafted starch, and isobutylene maleic anhydride copolymers, and mixtures of hydrolyzed acrylonitrile grafted starch, acrylic acid grafted starch, and isobutylene maleic anhydride copolymers.

Suitable high-absorbency materials are described in U.S. Pat. Nos. 4,699,823 issued Oct. 13, 1987, to Kellenberger et at. And 5,147,343 issued Sep. 15, 1992 to Kellenberger, which are incorporated herein by reference. High absorbency materials are available from various commercial vendors, such as Dow Chemical Company, Stockhausen, Inc., Hoechst Celanese Corporation, and Allied Colloids, Inc. In one aspect, the absorbent core 50 includes tissue layers or acquisition or distribution layers to maintain the integrity of fibrous absorbents or transport aqueous liquids.

The absorbent disposable underpant 12 includes additional components to assist in the acquisition, distribution, and storage of body exudates. In one aspect, the absorbent disposable underpant 12 includes a transport layer or surge layer intake material 114, such as described in U.S. Pat. No. 4,798,603 issued Jan. 17, 1989 to Meyer et al., or a surge management layer, such as described in U.S. Pat. Nos. 5,486,166 issued Jan. 23, 1996, to Bishop et al., 5,364,382 issued Nov. 15, 1994, to Latimer et al., 5,490,846 to Ellis et al., 5,429,629 to Latimer et al., 5,509,915 to Hanson et al., 5,192,606 to Proxmire et. al., and European Patent Application EP 0 539 703 A1, published May 5, 1993, which patents and application are incorporated herein by reference. Such layers are referred to as acquisition/distribution layers. A surge layer intake material 114 is positioned within about 0 inch (0 cm) to about 4 inches (10.2 cm) from the front end edge 55 of the absorbent core 50, from about 0 inch (0 cm) to about 2 inches (5.1 cm) from the front end edge 55 of the absorbent core 50 and more preferably from about 0 inch (0 cm) to about 1 inch (2.5 cm) from the front end edge 55 of the absorbent core 50.

The length of the surge layer (intake material) 114 is between about 5 inches (12.7 cm) and about 19 inches (48.3 cm), preferably between about 8 inches (20.3 cm) and about 16 inches (40.6 cm), and more preferably between about 10 inches (25.4 cm) and about 14 inches (35.6 cm). The length of the surge layer intake material 114 is about 12 inches (30.5 cm).

The acquisition/distribution layer 52 is disposed on the aqueous liquid storage layer 51 toward the body-facing surface 16 of the absorbent core 50 to decelerate and diffuse surges of aqueous liquid introduced into the absorbent core 50. The acquisition/distribution layer 52 includes a through-air bonded carded web composed of a blend of 40 percent 6 denier polyester fibers, available from Hoechst Celanese Corporation, and 60 percent 3 denier polypropylene/polyethylene sheath core bi-component fibers, available from the Chisso Corporation, with an overall basis weight ranging of from about 50 gsm and about 120 gsm.

Alternative acquisition/distribution materials are described in U.S. Pat. Nos. 5,192,606 issued Mar. 9, 1993, to D. Proxmire et al.; 5,486,166 issued Jan. 23, 1996 to Ellis et al.; 5,490,846 issued Feb. 13, 1996 to Ellis et al.; and 5,509,915 issued Apr. 23, 1996 to Hanson et al.; the disclosures of which are hereby incorporated by reference. The illustrated acquisition/distribution layer 52 is rectangular with a length of about 191 mm. And a width of about 45 mm. In one aspect, the acquisition/distribution layer 52 has a shape and size as disclosed for the absorbent core 50 and the disposable underpant 12.

A preferred absorbent core 50 includes a aqueous liquid storage layer 52 in which the basis weight of the absorbent components, such as fluff, pulp, and superabsorbent (SAP), are continuous throughout the MD length of the absorbent core 50. The distribution of the absorbent components are homogeneous in at least the Y direction, preferably in the X and Y directions and, in one aspect, homogeneous in the Z direction within the absorbent core 50. The basis weight of the absorbent core 50 ranges between about 80 gsm and about 1,000 gsm. More preferably, an acquisition/distribution layer 52 is disposed in the aqueous liquid storage layer 51, moved forward on the aqueous liquid storage layer 51. The fluff pulp/SAP ratio ranges from about 100:0 to about 40:60, and preferably from about 80:20 to about 50:50.

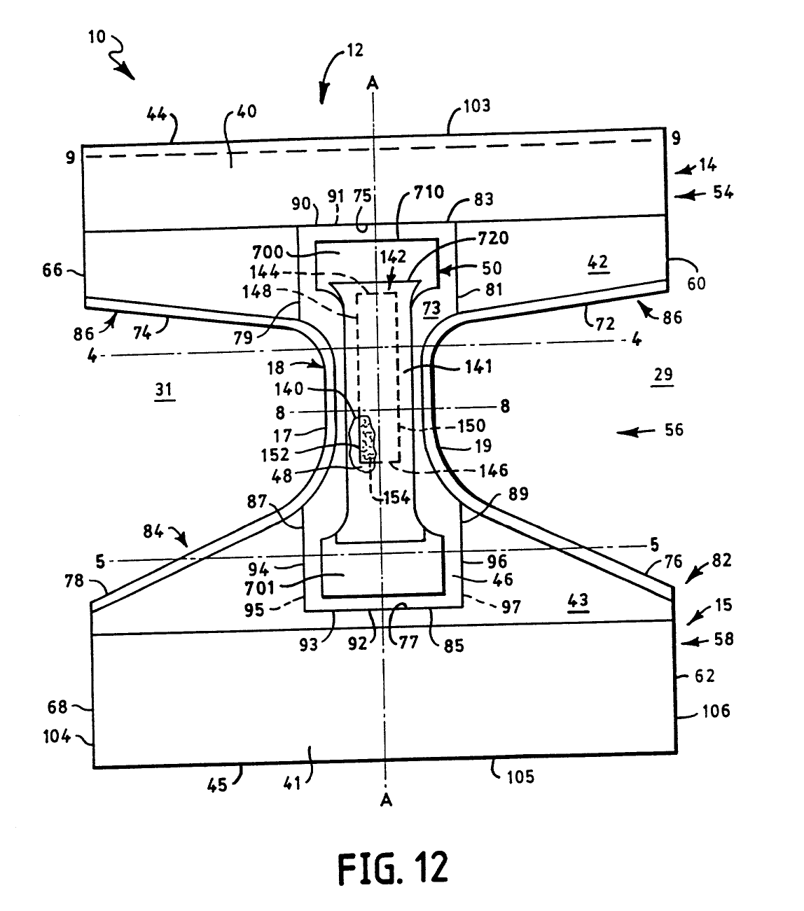

The pledget 140 has a peripheral edge 142 with a front edge 144, a back edge 146, and side edges 148 and 150 (See FIG. 12). Pledget 140 has an exterior surface 152 facing away from the wearer and an interior surface 154 facing towards the wearer. The pledget 140 has a dimension relative to the absorbent core 50 such that its width and length are each less than the width and length of the absorbent core 50, respectively. In this regard, the length of the pledget 140 is measured along the front and back edges 144 and 146 of the pledget 140, and the width of the pledget 140 is measured along the side edges 148 and 150 of the pledget 140. The length of the absorbent core 50 is measured between the front end edge 55 and the back end edge 57 and the width of the absorbent core 50 is measured between the side edges 59 and 61.

The pledget 140 preferably is made from a blend of fibers including about 15 to about 30 weight percent polypropylene or polyethylene fibers and about 85 to about 70 weight percent wood pulp fluff fibers, and has a basis weight of about 100 to about 525 gsm. In addition, a superabsorbent is added in an amount of about 10 to about 300 gsm. The pledget 140 is formed on a tissue or a spunbonded carrier sheet, or is formed without a carrier sheet.

The pledget 140 is illustrated in the drawings to have a dry thickness approximately equal to the dry thickness of the absorbent core 50. In one aspect, the pledget 140 and absorbent core 50 can have different thicknesses. In one aspect, the pledget 140 can be from about one-half to about four time the thickness of the absorbent core 50.

The pledget 140 can be positioned so as to be symmetrical about the central longitudinal axis A-A, B-B, respectively, of the disposable underpant 12. When in this position, the front and back edges 144 and 146 of pledget 140 are equidistant from the front edge 90 and the back edge 92 of the topsheet layer 49, respectively, and the side edges 148 and 150 of pledget 140 are equidistant from the side edges 94 and 96 of the topsheet layer 49, respectively. Further, when in this position, the absorbent core 50 extends past the peripheral edge 142 of the pledget 140.

The pledget 140, as illustrated in FIG. 12, can be positioned so that either the front or back edge 144 or 146 is no less than 2 inches (5.1 cm) from its respective front or back edges 90 and 92 of the topsheet layer 49 while still being symmetrical about the central longitudinal axis A-A. In other words, the pledget 140 can be asymmetrical about the transverse axis B-B.

In other embodiments of the present invention, the disposable underpant 12 includes a single layer absorbent core 50. The absorbent core 50 includes materials adapted to absorb and retain urine, menses, blood, or other body excrement. The absorbent core 50 includes various natural or synthetic absorbent materials, such as cellulose fibers, surfactant treated meltblown fibers, wood pulp fibers, regenerated cellulose or cotton fibers, or a blend of pulp and other fibers. One such material is coform material composed of a mixture of cellulosic fibers and synthetic polymer fibers. In one aspect, the absorbent core 50 includes compounds to increase absorbency, such as 0-95 weight percent of organic or inorganic high-absorbency materials capable of absorbing at least about 15 and up to more than 25 times their weight in water. Suitable high-absorbency materials are described in U.S. Pat. Nos. 4,699,823 issued Oct. 13, 1987, to Kellenberger et at. and 5,147,343 issued Sep. 15, 1992, to Kellenberger, which are incorporated herein by reference. High-absorbency materials are available from various commercial vendors, such as Dow Chemical Company, Hoechst Celanese Corporation, and Allied Colloids, Inc.

The absorbent core 50 provides the feature of being able to transport aqueous liquid in what can be characterized as in an x- and y-direction and in a z-direction. The transport of aqueous liquid in the z-direction is movement of a wicking nature and gravity flow where the aqueous liquid moves away from the body of the wearer. The transport of aqueous liquid in the x-direction and y-direction is movement and/or wicking of aqueous liquid along the length and width of the absorbent core 50. The movement of aqueous liquid both away from the wearer and along the length and width of the absorbent core 50 results in an increase in the utilization of the area of the absorbent core 50 because the aqueous liquid moves toward the distal ends of the absorbent core 50, and the result is an increase in the absorption of the absorbent core 50.

As illustrated in FIGS. 1a, 1b, 1c, 2a, 2b, 3, 10, 11, and 12, the absorbent core 50 has a width measured between the side edges 59 and 61 of the absorbent core 50. The absorbent core 50 has a length measured between the front end edge 55 and the back end edge 57 of the absorbent core 50. The width and length of the absorbent core 50 are each less than the corresponding width and length of the container 73 of the liquid barrier 48 and the aqueous liquid pervious topsheet layer 49. The width of container 73 is measured between the side edges 79 and 81 of container 73, and the length of the container 73 is measured between the front edge 75 and back edge 77. In one aspect, the width of the absorbent core 50 exceeds the width of the container 73 in an underpant product.

The overall length of the absorbent core 50 prevents aqueous liquid strike-through when sleeping or sitting.

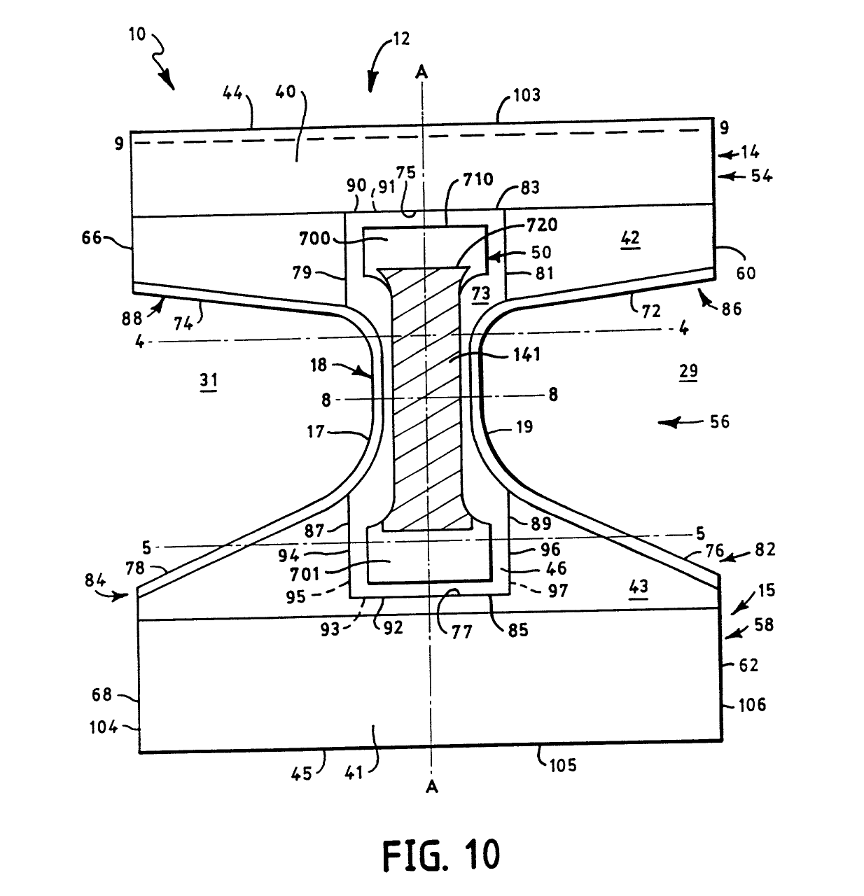

Referring now to FIGS. 10, 11, and 12, the central section 56 is the center one third of the total product length for the present invention, falling between lines 4-4 and 5-5. The front section 54 is the one third of the total product length of the disposable underpant 12 that falls between the line 4-4 and the front end edge 103 of the disposable underpant 12 and which is worn against the anterior side of the wearer's body. The back section 58 of the disposable underpant 12 is that one third of the length of the disposable underpant 12 between line 5-5 and the back end edge 105 of the disposable underpant 12 and is worn against the posterior side of the wearer's body. The overall length is at least about 10 inches (254 mm) thus extending beyond the central section 56 along the longitudinal centerline A-A of the disposable underpant 12. Alternatively, the length should be in the range of about 12 inches (305 mm) to about 30 inches (762 mm), preferably ranging from about 15 inches (381 mm) to about 23 inches (584 mm), more preferably from about 15 inches (381 mm) to about 21 inches (533 mm) in length, most preferably ranging from about 17 inches (432 mm) to about 20 inches (508 mm). In one aspect, the length of the absorbent core 50 is about 19 inches (483 mm).

The width of the absorbent core 50 extending beyond the crotch portion 18 is at least as wide as the width of the crotch portion 18. The width of the absorbent core 50 could be narrowed beyond the crotch portion 18 but may compromise the leakage containment. In some cases, the width of the absorbent core 50 is widened beyond the crotch portion 18, especially where the disposable underpant 12 includes leg cutouts 29 and 31 in the central section 56. Because the absorbent core 50 is disposed primarily in the front section 54, the central section 56, with less in the back section 58, a position shifted forward along the longitudinal axis of the disposable underpant 12, the leg cutouts 29 and 31 also are shifted forward along the longitudinal axis of the disposable underpant 12 to accommodate the position of the disposable underpant 12 on the body of the wearer. The width of the absorbent core 50 extending beyond the crotch portion 18 is from about 2.5 inches (64 mm) to about 12 inches (305 mm), preferably from about 4.0 inches (102 mm) to about 10 inches (254 mm), more preferably, from about 5 inches (127 mm) to about 9 inches (229 mm).

The present invention contemplates various shapes of the absorbent core 50. One preferred composite has a non-rectangular shape such as an hourglass or I-beamed shaped absorbent core 50.

Another preferred absorbent core 50 embodiment is rectangular in shape with rounded ends. The essentially rectangular-shaped absorbent core 50, i.e., an hourglass shape, is more preferred because it is squared off at the ends to provide a smoother appearance in the back of the disposable underpant 12 while providing a more comfortable body-contouring fit.

Absorbent core 50 is positioned so as to be symmetrical about the central longitudinal axis A-A of the underpant 12 and skewed forward along the central transverse axis B-B of the underpant 12. The side edges 59 and 61 of the absorbent core 50 are equidistant from side edges 94 and 96 of the aqueous liquid pervious topsheet layer 49, respectively. The front end and back end edges 55 and 57, respectively, of the absorbent core 50 are not equidistant from the front and back edges 103 and 105 of the aqueous liquid pervious topsheet layer 49, respectively. The absorbent core 50 is disposed in the front section 54, the central section 56, and the back section 58. The front end edge 103 of the absorbent core 50 is from about 10 inches (254 mm) to about 1 inch (25 mm), preferably from about 9 inches (229 mm) to about 2 inch (51 mm), more preferably from about 8 inches (203 mm) to about 3 inches (76 mm) from the front end edge 103 of the disposable underpant 12. In one preferred embodiment, the distance is about 3 inches (76 mm). The back end edge 105 of the absorbent core 50 is from 3 inches (76 mm) to about 13 inches (330 mm) preferably from about 5 inches (127 mm) to about 12 inches (305 mm), more preferably from about 6 inches (152 mm) to about 11 inches (279 mm) from the back end edge 105 of the disposable underpant 12.

Reference is made now to FIGS. 1a, 1b, 1c, 2a, 2b, 3, 10, 11, and 12 to describe a skewed forward absorbent core 50 along the central transverse axis. It is necessary in describing the present invention to define a “skew factor” of the absorbent core 50. The “skew factor” in combination with an absorbent core 50 in which more of the length of the absorbent core 50 is in the front section 54 than in the back section 58. The absorbent core 50 is not placed symmetrically in the longitudinal direction, but is skewed forward in the disposable underpant 12, and the “skew factor” defines the invention. For the purpose of this invention, “not placed symmetrically” means that more than about 20 mm or more of the absorbent core 50 is in the front section 54 as compared to the back section 58.

The skew factor is calculated using the following steps:

1. Divide the length of the disposable underpant 12 into three sections of equal length: the front section 54; the central section 56; and, the back section 58.

2. Determine what length in the longitudinal or MD direction of the absorbent core 50 along line A-A of FIGS. 10, 11, and 12 is in each section.

3. Calculate the skew factor by dividing the length of the absorbent core 50 in the back section 58 by the sum of the lengths of the absorbent core 50 in the front section 54 and the central section 56.

Referring to FIGS. 1a, 1b, 1c, 2a, 2b, 3, 10, 11, and 12, because absorbent disposable underpant 12 has a large range of product lengths to fit people from infants through adults, the skew factor in conjunction with the presence of more of the absorbent core 50 in the front section 54 than the back section 58 defines the amount of forward skew of an absorbent core 50 for any product length. The skew factor is a function of the overall length of the absorbent core 50 and how it is placed in disposable underpant 12. Because the overall length of disposable underpant 12 affects how much of the absorbent core 50 is in the front section 54, the central section 56, and the back section 58, the skew factor is also a function of the length of the disposable underpant 12. Placement of the absorbent core 50 in a range of product shapes and sizes of disposable underpants 12 depends on how that particular disposable underpant 12 is cut to fit on the wearer's body and the ability of the manufacturing process to control the placement of the absorbent core 50 within the disposable underpant 12. For the purposes of this invention, the skew factor is a value less than about 0.10. The skew factor ranges from above 0 to about 0.10, preferably from above 0 to about 0.095, more preferably from above 0 to about 0.085. In one aspect, the skew factors range from above 0 to about 0.07.

Referring now to FIGS. 10, 13a, and 14, in another embodiment, a disposable underpant 12 has a length of 845 mm, a width of 715 mm at the ends along line 9-9 and a minimum width of 120 mm in the central section 56 along line 8-8 and includes an absorbent core 50 having an MD length of 438 mm and a CD width of 153 mm at the ends and of 89 mm in the center of the leg cutout 29 and 31 along line 8-8. The absorbent core 50 is placed on aqueous liquid impervious barrier 48. The absorbent core 50 also has a high basis weight pocket region 141 which has a length of 279 mm, a width of 89 mm along line 8-8, and a width of 102 mm at the ends.

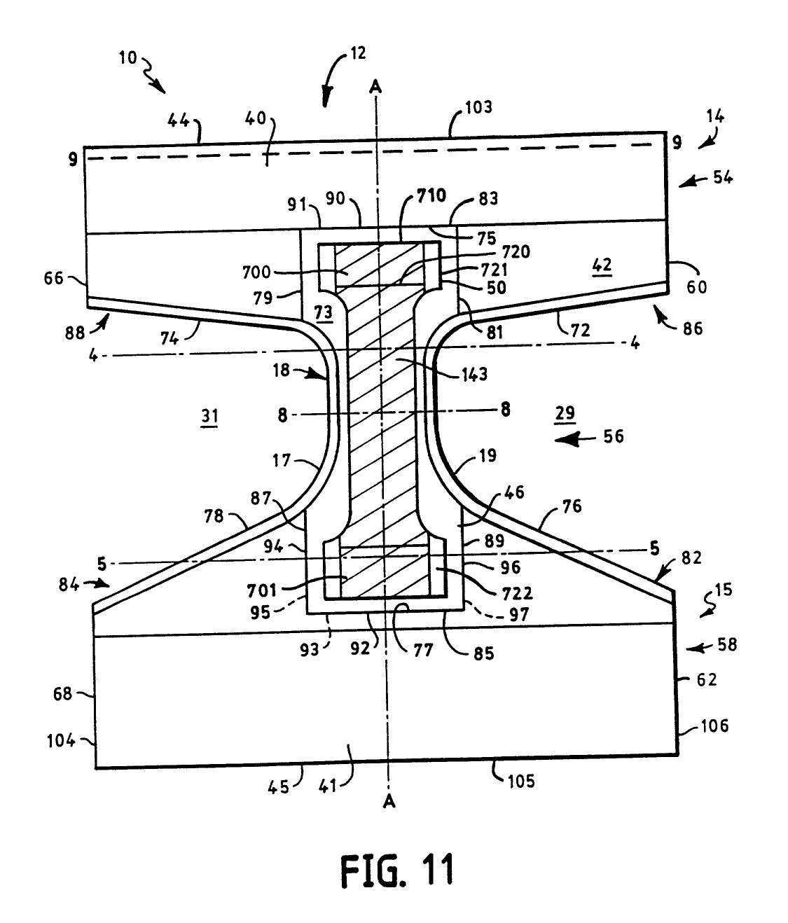

Referring now to FIGS. 13a and 14, the absorbent core 50 includes a homogeneous mixture of 129 gsm (grams per square meter) of DOW 2035 high absorbency material available from the DOW Chemical Company, Midland Mich. and 215 gsm of Alliance CR1654 fluff pulp fibers in the end regions 700 and 701. In the pocket region 141, the basis weight of the high absorbency material is 261 gsm and of the fluff pulp is 435 gsm. A carded web intake material 114 in the disposable underpant 12 is 330 mm long and 76 mm wide with a basis weight of 85 gsm comprising a mixture of 40% by weight 6 denier polyester fibers from Hoechst Celanese and 60% 3 denier sheath core polyethylene/polypropylene crimped fibers from CHISSO Corporation of Japan. The intake material 114 is located between the topsheet layer 49 and the absorbent core 50. The absorbent core 50 has a retention capacity of about 500 grams of 0.9% sodium chloride in water. Importantly, the absorbent core 50 is placed in disposable underpant 12 so that the front end edge 55 of absorbent core 50 is 163 mm from the front end edge 103 of disposable underpant 12. Furthermore, the front end edge 720 of the pocket region 141 is placed 222 mm from front end edge 103 of the disposable underpant 12. Finally, an 85 gsm surge material intake material with a length dimension of 279 mm and a width of 64 mm is placed coextensive with the pocket region 141 in the length dimension and centered in the width dimension on the interior surface 65 of the absorbent core 50. The absorbent core 50 has a retention capacity of about 500 grams of 0.9% sodium chloride in water. A measure of 27.1% of the length of the absorbent core 50 in the machine direction is placed in the front section 54 of the disposable underpant 12, 64.2% of the length of the absorbent core 50 is in the central section 56 of the disposable underpant 12, and 8.7% of the length of the absorbent core 50 is in the back section 58 of the disposable underpant 12. The absorbent core 50 has a skew factor of 0.095. Additionally, 21.5% of the length of the pocket area 141 is in the front section 54, 78.5% of the length of the pocket area 141 is in the central section 56, and 0.0% of the length of the pocket area 141 is in the back section 58. The absorbent core 50 and the intake material 114 placement is symmetric in the CD dimension of the disposable underpant 12 along the central longitudinal axis A-A in FIG. 11. When placed on the body of the wearer, the preferred configuration results in a greater proportion of the absorbent core 50 being on the anterior side of the wearer where it is more likely to be used.

Compared to symmetric placement of an absorbent pad or core in the MD length of a disposable underpant resulting in equal placement of the absorbent pad or core in the anterior and posterior portions of such a disposable underpant, the skewed forward configuration results in higher utilization of the absorbent core 50 and prevents unsightly and uncomfortable bulk in the back section 58 of the disposable underpant 12.

Referring now to FIGS. 11, 13a, and 14 in yet another embodiment, a disposable underpant 12 has a length of 845 mm, a width of 715 mm at the ends along the line 9-9, and a minimum width of 120 mm in the central section 56 along line 8-8. An absorbent core 50 has an MD length of 489 mm and a CD width of 153 mm at the ends and of 89 mm in the center of the leg cutout 29 and 31 along line 8-8. The absorbent core 50 is placed on the aqueous liquid impervious barrier 48. The absorbent core 50 has a high basis weight pocket region 143 which has a length of 489 mm full length of the absorbent core 50, a width of 89 mm along line 8-8, and a width of 102 mm at the ends.

Referring now to FIGS. 13b and 14, the absorbent core 50 includes a homogeneous mixture of 114 gsm (grams per square meter) of DOW 2035 high absorbency material available from the DOW Chemical Company, Midland Mich. and 232 gsm of Alliance CR1654 fluff pulp fibers in the side end regions 721 and 722. In the pocket region 143, the basis weight of the high absorbency material is 282 gsm and of the fluff pulp is 591 gsm. The absorbent core 50 has a retention capacity of about 800 grams of 0.9% sodium chloride in water. Importantly, the absorbent core 50 is placed in the disposable underpant 12 so that the front end edge 55 of absorbent core 50 is 112 mm from the front end edge 103 of the disposable underpant 12. Finally, an 85 gsm surge material intake material with a length dimension of 279 mm and a width of 64 mm is placed 187 mm from the front end edge 103 of the disposable underpant 12 and is centered in the width dimension along the central longitudinal axis A-A placed on the interior surface 65 of the absorbent core 50.

A measure of 34.7% of the length of absorbent core 50 in the machine direction is placed in the front section 54 of the disposable underpant 12. A measure of 57.6% of the length of the absorbent core 50 is in the central section 56 of the disposable underpant 12, and 7.7% of the length of the absorbent core 50 is in the back section 58 of the disposable underpant 12. The absorbent core 50 has a skew factor of 0.082. The absorbent core 50 and the intake material 114 placement are symmetrical in the CD dimension of the disposable underpant 12 along the central longitudinal axis A-A. When placed on the body of the wearer, the configuration results in a greater proportion of the absorbent core 50 being on the anterior side of the wearer where it is more likely to be used.

Compared to symmetric placement of an absorbent pad or core in the MD length of a disposable underpant resulting in equal placement of the absorbent pad or core in the anterior and posterior portions of such a product, the skewed forward configuration results in higher utilization of the absorbent core 50 and prevents unsightly and uncomfortable bulk in the back section 58 of the disposable underpant 12.

### EXAMPLE

Two absorbent disposable underpant products were tested for leakage protection performance. The disposable underpants were sent to 72 incontinent panelists (44 females, 28 males) who used them under normal use conditions. A diary sheet, with pre-written questions, was provided for all individual products on which panelists recorded information pertaining to leakage. All used products were returned and weighed to determine the amount of urine they contained.

From the combined data, a stepwise procedure for logistic regression was used to determine the best fitting model for the leakage data. The goal of logistic regression was to describe the relationship between leaks and the set of explanatory variables of codes, urine grams, gender, activity, and panelist hip size.

The disposable underpant products tested were a prototype disposable underpant and the SureCare® Slip-On Undergarment produced by Inbrand Corporation of Marietta, Ga. The Slip-on product is a traditional pant with a symmetrical placement of the absorbent core in the product chassis while the prototype disposable underpant had a highly skewed forward absorbent core with less capacity than the SureCare product.

The SureCare product was 660 mm long and had an essentially rectangular absorbent core which was 550 mm long and 127 mm wide. The absorbent core had 90 gsm of high absorbency material and 1062 gsm of fluff pulp. The absorbent core was placed 55 mm from the front and back end edges of the garment.

The prototype disposable underpant, referring to FIG. 10, had a product length of 781 mm. The absorbent core was 400 mm long and 165 mm wide at the ends and 90 mm wide in the center. The absorbent core was placed 160 mm from the front end edge of the prototype disposable underpant and 221 mm from the back end edge of the prototype underpant. The absorbent core included 110 gsm of high absorbency material and 165 gsm of fluff fibers.

The prototype disposable underpant had a pledget which was 292 mm long, 90 mm wide and placed 185 mm from the front end edge of the prototype underpant and 304 mm from the back end edge of the prototype underpant between the absorbent core and the liquid barrier. The pledget comprised of 180 gsm of the high absorbency material and 270 gsm of the fluff fibers. The density of the absorbent core and the pledget was about 0.160 gm/cc.

The prototype disposable underpant had a 100 gsm intake material which was 64 mm wide and 203 mm long. The front end edge of the intake material was 216 mm from the front end edge of the prototype disposable underpant and 362 mm from the back end edge of the prototype disposable underpant.

The proportions of the absorbent core and the pledget in the front section, central section, and back section of the prototype disposable underpant are shown in Table I. Table II shows that the absorbent core, the pledget, and the intake material of the prototype disposable underpant were skewed to the front of the prototype disposable underpant with higher proportions of the absorbent core in the front while the SureCare product had a symmetric placement of its absorbent core.

TABLE I
Dimensional Comparison of Prototype Disposable
Underpant and SureCare Slip-On

| | | | | | Reten- |
|---|---|---|---|---|---|
| | | | | | tion |
| | | % in | % in | % in | Capacity |
| Pro- | Absorbent | Front | Central | Back | 0.9% |
| duct | Component | Region | Region | Region | Saline |
| Sure- | Absorbent | 30.0 | 40.0 | 30.0 | 726 g |
| Care | Core | | | | |
| Proto- | Absorbent | 25.0 | 65.0 | 9.8 | 480 g |
| type | Core | | | | |
| Dispos- | Pledget | 25.7 | 74.3 | 0.0 | |
| able | | | | | |
| Under- | Intake | 21.2 | 78.8 | 0.0 | |
| pant | Material | | | | |

The SureCare product in TABLE I did not have a skew factor because the placement of the absorbent core was symmetric. On the other hand, the skew factor of the absorbent core of the prototype disposable underpant was 0.111, showing the absorbent core to be highly skewed forward.

The leakage information in TABLE II, expressed as the urine load in grams at which 20% (LD20) and 50% (LD50) of the products leak clearly shows that the prototype disposable underpant with the skewed forward absorbent core provides preferred leakage protection because a higher urine load is needed to make 20% and 50% of the products leak.

TABLE II
Leakage Protection of SureCare Slip-on versus Prototype Disposable
Underpant

| Product | Gender | LD20, grams | LD50, grams |
|---|---|---|---|
| SureCare | Male | 240 | 367 |
| | Female | 84 | 231 |
| Prototype Dis- | Male | 294 | 450 |
| posable | | | |
| Underpant | Female | 294 | 441 |

While specific embodiments of a disposable underpant 12 are shown in the Figures, it is understood that it is contemplated that any disposable underpant product can incorporate the absorbent core 50 being placed such that the product would have a skew factor as specified in accordance with the article and method of the present invention as disclosed and claimed.

Preferably, the thickness of the absorbent core 50 is less than about 45 mm. The thickness of the absorbent core 50 ranges from about 4 mm to about 40 mm. Preferably, the thickness ranges from about 5 mm to about 30 mm with a thickness having a more preferred range from about 6 mm to about 20 mm. The thickness is measured on a 4 inch (102 mm) smooth unbuckled square sample, elastics removed, with a Mitutoyo Digamatic Indicator using a 3 inch (76 mm) diameter acrylic platen and assembly to produce a pressure of 0.05 psi.

In construction of the absorbent barrier composite 46, the liquid barrier 48 should retard the movement of the liquid through the absorbent barrier composite 46 by making the liquid barrier 48 resistant to penetration normally encountered under wearing conditions. The absorbent barrier composite 46 is rendered liquid impermeable by coating the absorbent core 50 or by securing a separate liquid impermeable material to the absorbent core 50.

The liquid barrier 48 has an exterior surface 99 facing away from the wearer and an interior surface 98 facing toward the wearer. The liquid barrier 48 includes a thin, liquid impervious web or sheet of plastic film such as polyethylene, polypropylene, polyvinyl chloride, or similar material. In one embodiment, the liquid barrier 48 includes a nonwoven, fibrous web constructed and arranged to have low liquid perviousness. In one embodiment, the liquid barrier 48 includes a layered or laminated material, such as a thermally bonded plastic film and nonwoven web composite. In one embodiment, the liquid barrier 48 includes a liquid impervious film or foam which is pervious to water vapor under normal wearing conditions. The liquid barrier 48 preferably has a water vapor transmission rate of at least about 1000 grams/m²/day measured by ASTM E96-92. One example of a suitable film is a 39.4 grams per square meter microporous film produced by Mitsui and sold by Consolidated Thermoplastics (CT) under the tradename of ESPOIR® N-TAF-CT.

The liquid barrier 48 includes a material formed or treated to be fluid impermeable. In one aspect, the liquid barrier 48 includes a fluid permeable material and other means such as a fluid impermeable layer associated with the absorbent barrier composite 46 or the absorbent core 50, provided to impede fluid movement away from the absorbent barrier composite 46 or the absorbent core 50. The liquid barrier 48 includes a single layer of material or a laminate of two or more separate layers of material. Preferred materials include a single spunbonded layer, two layers of spunbonded and meltblown materials, or three-layer material of spunbonded-meltblown-spunbonded material.

Materials for the liquid barrier 48 include thermoplastic films, wovens, nonwovens, laminates of films, wovens, and/or nonwovens, or foams. The liquid barrier 48 includes a thin, substantially fluid impermeable web or sheet of plastic film such as polyethylene, polypropylene, polyvinyl chloride, or similar material. One material for the liquid barrier 48 is a 0.028 millimeter (mm) thick polyethylene film with a systematic matte embossed pattern and that has been corona treated on both sides.

The liquid barrier 48 prevents liquid strike-through to the outer clothing when discharge occurs onto the absorbent barrier composite 46 of the underpant 12. The liquid barrier 48 is located on the inside of the outer cover 13 in at least the crotch portion 18 and includes a liquid impervious film such as polyethylene. Use of only the film without the outer cover 13 is hot and uncomfortable and is not durable enough to withstand extended periods of wear. The absorbent core 50 is associated with a liquid barrier 48 which may or may not include elastic characteristics.

The absorbent barrier composite 46 includes a substantially fluid impermeable liquid barrier 48, a fluid (or liquid) permeable topsheet layer 49 superposed on the outer cover 13. (See FIGS. 1a, 8, and 9.) The absorbent core 50 is sandwiched between the liquid barrier 48 and the topsheet layer 49. (See FIGS. 1a, 8, and 9.) The liquid barrier 48 and the topsheet layer 49 are longer and wider than the absorbent core 50. The topsheet layer 49 is positioned toward the wearer and is referred to as the body-facing surface 16. Conversely, the liquid barrier 48 is positioned toward the outer cover 13 and the outer clothing of the wearer and is referred to as the garment-facing surface 23.

The topsheet layer 49 has an exterior surface 109 facing away from the wearer and an interior surface 108 facing toward the wearer. The topsheet layer 49 includes a nonwoven or other soft material for contacting the wearer's skin. The topsheet layer 49 is formed of a soft flexible porous aqueous liquid pervious material so that aqueous liquid waste, and possibly semi-solid waste, passes through to the absorbent core 50 and is absorbed by the absorbent barrier composite 46 or absorbent core 50. A suitable topsheet layer 49 includes a nonwoven web, a spunbond, meltblown or bonded-carded web composed of synthetic polymer filaments or fibers, such as polypropylene, polyethylene, polyesters, a perforated film, reticulated foams, an expanded plastic webbing material, scrim material, or a web or natural polymer filaments or fibers such as wood, rayon, or cotton. The topsheet layer 49 has a pore size allowing the passage of air, sweat, or perspiration because of the breathability of the material.

The topsheet layer 49 is a nonwoven spunbond. Such spunbond material is available from Kimberly-Clark Corporation, located in Roswell, Ga. The topsheet layer 49 has a weight from about 0.3 osy to about 2.0 osy and alternatively about 0.5 osy. The topsheet layer 49 of the underpant is printed, colored, decoratively embossed, or perforated with discrete slits or holes.

In addition, the topsheet layer 49 is treated with a surfactant to aid in fluid transfer. In one embodiment, the top-sheet layer 49 includes a nonwoven, spunbond web of sheath core bicomponent filaments with 50 percent polyethylene and 50 percent polypropylene having a basis weight of about 20 grams per square meter (gsm). The fabric is surface treated with a surfactant available from Union Carbide Chemicals and Plastics Company, Inc., of Danbury, Conn., U.S.A. under the trade designation TRITON X-102.

The width of the crotch portion 18 between the crotch elastics 37 and 39 is wide enough to accommodate the absorbent core 50 between the side edges 17 and 19 of the crotch portion 18 without having the absorbent core 50 obstruct the crotch elastics 37 and 39 and ultimately the leg elastics 32, 33, 34, and 35. The width of the crotch portion 18 allows the crotch elastics 37 and 39 to contract and draw up the sides of the crotch creating a bucket with walls of the topsheet layer 49 and liquid barrier 48 to keep bodily exudates from leaking out of the product and to accommodate more sizes of individuals. In one embodiment, the crotch elastics 37 and 39 are covered by at least a portion of the absorbent core 50.

The width of the crotch portion 18 is not so wide as to seem bulky or uncomfortable, but a suitable width is at least about 2.5 inches (64 mm) between the crotch elastics 37 and 39. The width ranges from about 2.5 inches (64 mm) to about 6.0 inches (152 mm). The width of the crotch portion 18 between the crotch elastics 37 and 39 ranges from about 3.5 inches (89 mm) to about 5 inches (127 mm). Preferably, the width so defined is about 4.25 inches (108 mm).

The crotch portion 18 is at least about 0.25 inch (6 mm) wider than the width of the absorbent core 50. The crotch portion 18 is from about 0.25 inch (6 mm) to about 4 inches (102 amm) wider than the absorbent core 50. The crotch portion 18 is from about 0.5 inch (13 mm) to about 3 inches (76 mm) wider than the absorbent core 50 and preferably from about 0.5 inch (13 mm) to about 2 inches (51 mm) wider. Preferably, each of the crotch elastics 37 and 39 are from about 0.2 inch (5 mm) to about 0.8 inch (20 mm) wide. More preferably, the width of each crotch elastics 37 and 39 is from about 0.2 inch (5 mm) to about 0.4 (10 mm). The overall width of the crotch portion 18 includes the width between the crotch elastics 37 and 39, the width of the crotch elastics 37 and 39, and the ruffle material outside the crotch elastics 37 and 39 to the edge of the leg openings 28 and 30. In one embodiment of the present invention, the absorbent core 50 is from about 0 inches (0 mm) to about 1 inch (25 mm) wider than the crotch portion 18.

Preferably, ruffle material on the edge of the leg openings 28 and 30 outside the leg and crotch elastics 32, 33, 34, 35, 37, and 39 is less than about 0.5 inch (13 mm). More preferably, the ruffle material is less than about 0.125 inch (3 mm). It is preferred to eliminate the ruffle material from the edge of the leg openings 28 and 30.

The width of the absorbent barrier composite 46 is sized in relation to the width of the crotch portion 18. The width of the composite 46 is at least the width of the crotch portion 18 between the crotch elastics 37 and 39. Preferably, the width is equivalent to the width of the crotch portion 18.

The overall length of the absorbent barrier composite 46 is adequate to extend beyond the ends of the absorbent core 50 to help prevent liquid strike through at these points when sleeping or sitting. This overall length is at least about 12 inches (305 mm) thus extending beyond the crotch portion 18 along the longitudinal centerline A-A of the underpant 12. Alternatively, the length is in the range of about 12 inches (305 mm) to about 30 inches (762 mm), preferably ranging from about 15 inches (381 mm) to about 23 inches (584 mm). A common range is from about 17 inches (432 mm) to about 21 inches (533 mm) in length. Optimally, the length of the composite 46 is about 19 inches (483 mm).

The width of the absorbent barrier composite 46 extending beyond the crotch portion 18 is at least as wide as the width of the crotch portion 18. The width of the absorbent barrier composite 46 could be narrowed beyond the crotch portion 18 but may compromise the leakage containment. The width of the absorbent barrier composite 46 is widened beyond the crotch portion 18. The width of the absorbent barrier composite 46 extending beyond the crotch portion 18 is from about 2.5 inches (64 mm) to about 12 inches (305 mm), preferably from about 4.0 inches (102 mm) to about 10 inches (254 mm). A preferred range is from about 7 inches (178 mm) to about 9 inches (229 mm). A preferred width is about 8 inches (203 mm).

The present invention includes different shapes of the absorbent barrier composite 46. One preferred composite has a non-rectangular shape such as an hourglass or I-beamed shaped absorbent barrier composite 46 which provides extensive coverage in the seat of the finished underpant 12. Another preferred absorbent barrier composite 46 embodiment is rectangular in shape with rounded ends. The essentially rectangular-shaped absorbent barrier composite 46, i.e., an hourglass shape, is more preferred since it can be squared off at the ends to provide a smoother appearance in the back of the underpant 12 while providing a more comfortable body-contouring fit.

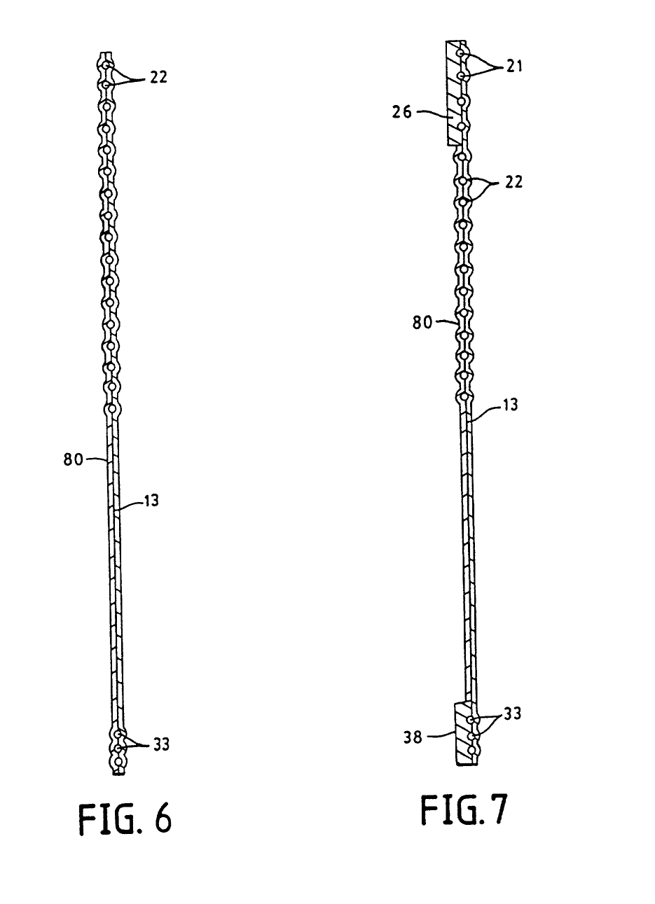

Referring to FIGS. 1a-1c, 2a-2b, 3, and 6, the waist portion elastic 22 are shown covered with a body liner 80. The body liner 80 includes a nonwoven or other soft material for contacting the wearer's skin. The body liner 80 is described in more detail below. In other embodiments, at least a portion of the waist portion elastic 22 is covered by the absorbent barrier composite 46. Referring to FIGS. 3 and 4, the leg elastics 32, 33, 34, and 35 are shown covered by the body liner 80. In FIG. 1b, the crotch elastics 37 and 39 are shown sandwiched between the topsheet layer 49 and the liquid barrier 48. In alternative embodiments, the crotch elastics 37 and 39 can be sandwiched between the outer cover 13 and the liquid barrier 48. Depending on the size and shape of the absorbent barrier composite 46, at least a portion of the leg elastics 32, 33, 34, and 35 is covered by the absorbent barrier composite 46.

Referring to FIGS. 5 and 7, the waist elastic 21 are shown in another embodiment as covered with a waist liner 26. The waist elastic 21 could also be covered with the body liner 80. Referring to FIGS. 5 and 7, the leg elastics 32 and 34 are shown covered by a leg liner 38. Depending on the size and shape of the absorbent barrier composite 46, at least a portion of the leg elastics 32, 33, 34, and 35 is covered by the absorbent barrier composite 46. In FIG. 1a, the crotch elastics 37 and 39 are shown covered by the absorbent barrier composite 46 as the crotch elastics 37 and 39 are sandwiched between the outer cover 13 and the absorbent barrier composite 46. Referring to FIG. 7, the waist portion elastic 22 are shown covered with a body liner 80. The body liner 80 includes a nonwoven or other soft material for contacting the wearer's skin. In one embodiment, at least a portion of the waist portion elastic 22 is covered by the absorbent barrier composite 46.

The body liner 80 is compliant and soft feeling to the wearer. The body liner 80 is a soft, flexible, porous sheet which is liquid pervious, permitting liquids to penetrate readily into its thickness, or impervious, resistant to the penetration of liquids into its thickness. The body liner 80 is manufactured from a materials such as natural fibers, e.g., wood or cotton fibers) synthetic fibers, e.g., polyester or polypropylene fibers, or from a combination of natural and synthetic fibers or reticulated foams and apertured plastic films.

The body liner 80 is woven or nonwoven web or sheet such as a spunbond, meltblown, or bonded-carded web composed of synthetic polymer filaments, such as polypropylene, polyethylene, polyesters, or a web of natural polymer filaments such as rayon or cotton. The bonded-carded web is thermally bonded or sprayed with a binder. The body liner 80 is a nonwoven spunbond. The body liner 80 is a spunbond polypropylene nonwoven with a wire-weave bond pattern. The spunbond material is available from Kimberly-Clark Corporation, located in Roswell, Ga. The body liner 80 has a weight from about 0.3 oz. per square yard (osy) to about 2.0 osy and alternatively about 0.6 osy. The body liner 80 of the underpant may be printed, colored or decoratively embossed. The body liner 80 has a pore size that readily allows the passage of air, sweat, perspiration because of the breath-ability of the material. The body liner 80 is selectively embossed or perforated with discrete slits or holes.

The position and the shape of the leg openings 28 and 30 are important to avoid tightness in the crotch and groin area of the wearer, to obtain adequate buttocks coverage, and to prevent the underpant 12 from tilting forward, i.e., tilting such that the front waist edge dips lower in relationship to the back waist edge. FIG. 1a illustrates the most preferred design for leg fit and buttocks coverage. The shape of the curve across the front edges 72 and 74 of the leg openings 28 and 30 has an effect on the fit of the underpant 12. If the curve is too deep, the underpant 12 will shift downward and backward resulting in a short front waist, increased back length, and bagginess in the seat of the underpant 12. The shifting causes the underpant 12 to appear tilted when worn as evidenced by an unevenness around the waist of the wearer, resulting in tightness along the front edges 72 and 74 of the leg openings 28 and 30 and reducing the comfort in the fit.

The structures of the leg openings 28 and 30 are important to the correct functioning of the underpant 12. With the underpant 12 laid out flat as in FIG. 1a, the majority, i.e., at least 51%, of the back edges 76 and 78 of the back portions 82 and 84 of the leg openings 28 and 30 respectively preferably forms a substantially linear line. More preferably, the back edges 76 and 78 of the back portions 82 and 84 of the leg openings 28 and 30 are substantially linear for a length, Θ, of at least about 70% of the length of the entire back portions 82 and 84. The straight section Θ of the back edges 76 and 78 of the back portions 82 and 84 of the leg openings 28 and 30 form an acute angle with the longitudinal centerline, A-A, of the underpant 12. Preferably, the line, Θ, forms an angle, α, with the centerline A-A of the underpant 12 of between about 45° and about 89°, more preferably between about 55° and about 87° and most preferably between about 61° and about 76°. If a shallow curve, i.e., a curve having a radius of at least 6 inches, defines the back edges 76 and 78 of the back portions 82 and 84 of the leg openings 28 and 30, two points are selected along the curve, one point is positioned at about 25% of length of the curve and the other point is positioned at about 75% of the length of the curve. A line drawn between the two points is used to determine the α angle formed with the centerline A-A of the underpant 12. The back edges 76 and 78 forming acute angles with the longitudinal centerline as disclosed reduce bunching and shifting of the absorbent core 50 during use, ensuring a better contouring to the body and comfortable fit. Such angles help the underpant 12 to undercut the buttocks, improving fit and reducing leakage. Because the leg elastics 34 and 35 closely follow the back edges 78 and 76, respectively, the references to the back edges 78 and 76 can be read to describe the leg elastics 34 and 35.

The majority, i.e., at least 51%, of the front edges 72 and 74 of the front portions 86 and 88 of the leg openings 28 and 30 include lengths β, preferably forming straight lines. More preferably, the lengths of front edges 72 and 74 (β) of the leg openings 28 and 30 are linear for at least about 70% of the length of the front portions 86 and 88 of the leg openings 28 and 30. The linear section β of the front edges 72 and 74 of the front portions of the leg openings 28 and 30 form an angle χ with the centerline of the underpant 12 of between about 62° and about 99°, more preferably between about 74° and about 91°, and most preferably about 85°. The front edges 72 and 74 forming an angle with the centerline as disclosed reduce bunching and shifting of the absorbent core 50 during use, ensuring a better contouring to the body and comfortable fit. If a shallow curve, i.e., a curve having a radius of at least 6 inches, defines the front edges 72 and 74 of the front portions 86 and 88 of the leg openings 28 and 30, two points are selected along the curve, one point is positioned at about 25% of length of the curve and the other point is positioned at about 75% of the length of the curve. A line drawn between the two points is used to determine the χ angle formed with the centerline of the underpant 12. While front edges 72 and 74 forming angles of 90° can be used, angles at least slightly greater or less than 90° provide an underpant 12 having a comfortable fit during movement. Because the leg elastics 32 and 33 closely follow the front edges 72 and 74 respectively, the references to the front edges 72 and 74 are read to describe the leg elastics 32 and 33.

The shape of the curve in the crotch portion 18 is important. The majority of the side edges 17 and 19 of the crotch portion 18 include lengths Σ, preferably forming curved arcuate lines. If the curve is too shallow or straight especially at the ends of the crotch elastics 37 and 39, tightness is experienced at the inner groin area and gapping in the fit of the underpant 12 as the underpant 12 does not follow the contour of the wearer's leg, resulting in leakage. The preferred narrow crotch width having shallow curves may also reduces the coverage of the buttocks. To compensate for such reduction, the back leg edges 76 and 78 on the back portions 82 and 84 are preferably adjusted downward, increasing the α angle. The curve between the side edges 17 and 19 of the crotch portion 18 and the back portions 82 and 84 of the leg openings 28 and 30 should start slightly in front of centerline B-B of the underpant 12, see FIG. 1a. Accordingly, the back leg elastics 34 and 35 are positioned below the lower edge of the buttocks and prevent the underpant 12 from riding up when walking. The curved crotch portion Σ of the leg openings 28 and 30 are entirely forward of the underpant 12 centerline B-B. Because the crotch elastics 37 and 39 closely follow the edges 17 and 19 respectively, the references to the edges 17 and 19 are read to describe the leg elastics 37 and 39.

While the leg elastics 32, 33, 34, and 35 closely follow the edge of the absorbent core 50 outside of the crotch portion 18, moving the leg elastics 32, 33, 34, and 35 away from the absorbent core 50, the absorbent core 50 interferes less with the function of the leg elastics 32, 33, 34, and 35, providing better gasketing around the leg openings 28 and 30. Absorbent core 50 swells as it absorbs bodily discharges, and the leg elastics 32, 33, 34, and 35 are so positioned to remain in contact with and conformed to the wearer's body.

In a preferred embodiment, the waist portion and leg elastics 22, 32, 33, 34, and 35, respectively, and in embodiments including waist elastic 21 are attached to the underpant 12 sandwiched between the outer cover 13 and the body liner 80 in a stretched state by ultrasonic bonded, heat/pressure bonded, or adhesively bonded. The crotch elastics 37 and 39 are sandwiched between the liquid barrier 48 and the topsheet layer 49 of the absorbent barrier composite 46. The crotch elastics 37 and 39 are, attached in a stretched state by ultrasonic bonded, heat/pressure bonded, or adhesively bonded. Materials for the elastics include elastic strands, yarn rubber, flat rubber, elastic tape, film-type rubber, polyurethane and elastomeric, tape-like elastomeric, or foam polyurethane or formed elastic or non-elastic scrim. Suitable material is sold under the name LYCRA® by the DuPont Company located in Wilmington, Del. Each elastic is unitary, multi-part, or composite in construction before integrating into the underpant 12.

In an alternative embodiment, the waist portion, leg, and crotch elastics 22, 32, 33, 34, 35, 37, and 39, respectively, and in embodiments including waist elastic 21 are attached to the underpant 12 sandwiched between the outer cover 13 and the body liner 80 in a stretched state by ultrasonic bonded, heat/pressure bonded, or adhesively bonded. Materials for the elastics include elastic strands, yarn rubber, flat rubber, elastic tape, film-type rubber, polyurethane and elastomeric, tape-like elastomeric or foam polyurethane or formed elastic or non-elastic scrim. Suitable material is sold under the name LYCRA® by the DuPont Company located in Wilmington, Del. Each elastic is unitary, multi-part, or composite in construction.

The portion of the front and back body portions 14 and 15 having waist portion elastic 22 is from about 1 inch (25 mm) to about 10 inches (254 mm) wide (perpendicular to A-A as shown in FIG. 1a). Preferably, the portion of the front and back body portions 14 and 15 having waist portion elastic 22 is from about 3 inches (76 mm) to about 8 inches (203 mm) wide. Preferably, the width ranges from about 6 inches (152 mm) to about 7.5 inches (191 mm). In one embodiment, the widths of waist portion elastic 22 vary between the front and back body portions 14 and 15. If one of the body portions 14 and 15 has a narrower portion of waist portion elastic 22, the width ranges between about 1 inch (25 mm) to about 8 inches (203 mm), preferably from about 3 inches (76 mm) to about 6 inches (152 mm), and more preferably from about 4 inches (102 mm) to about 5.5 inches (140 mm). The waist portion elastic 22 is applied under an elongation of from about 100% to about 400%, preferably under an elongation of from about 150% to about 300%, and more preferably under an elongation of from about 200% to about 275%. The underpant 12 is constructed such that the tension of the waist portion elastic 22 is a different value in the front body portion 14 as compared to the tension of the waist portion elastic 22 in the back body portion 15.

The waist portion elastic 22 includes threads, strands, ribbons, bands, film, elastic nonwovens, or composite. The threads, strands, ribbons, or bands are multiple and are applied as a composite. The number of pieces of elastic material comprising the waist portion elastic 22 ranges from about 1 to about 100, preferably from about 10 to about 40, and more preferably from about 15 to about 35. Preferably, when the waist portion elastic 22 are threads, 15 to 40 threads are used as the waist portion elastic 22, and the threads are spaced from about 0.0625 inch (1.6 mm) to about 2 inches (51 mm), more preferably from about 0.0625 inch (1.6 mm) to about 0.5 inch (13 mm), and most preferably about 0.25 inch (6 mm) apart. While the appearance of the underpant 12 is enhanced by the close even spacing of the elastic material, such as threads, in the waist portion elastic 22, however, the distance between the pieces of elastic material can be varied. Such variation in the front and back waist edge portions 44 and 45 give the appearance of a waist band.

In a preferred embodiment, the elastic threads are made of elastomeric material. One material is spandex such as LYCRA® threads available from DuPont located in Wilmington, Del. Waist portion elastic 22 includes threads having a decitex (g/10000 m) of about 100 to about 1200, preferably from about 470 to about 940, and more preferably from about 620 to about 740 for waist portion elastic 22 comprising from about 15 to about 35 threads. Adhesive 71, applied in a meltblown or swirl pattern, is used to bond the waist portion elastic 22 to the outer cover 13 and the body liner 80. Preferably, the adhesive 71 is applied only to the waist portion elastic 22. A suitable adhesive includes, for example, Findley H2096 hot melt adhesive available from Ato Findley Adhesives located in Milwaukee, Wis.

In embodiments that include waist elastic 21, the waist elastic 21 is from about 0.0625 inch (1.6 mm) to about 2 inches (51 mm) wide. Preferably, the front and back waist edge portions 44 and 45 of the front and back body portions 14 and 15, respectively, having waist elastic 21 from about 0.25 inches (6 mm) to about 1.5 inch (38 mm) wide. Preferably, the width ranges from about 0.5 inches (13 mm) to about 1 inches (25 mm). The waist elastic 21 is applied under an elongation of from about 150% to about 300%, preferably under an elongation of from about 175% to about 275%, and more preferably under an elongation of from about 200% to about 250%.

The waist elastic 21 includes threads, strands, ribbons, film, elastic nonwovens, or composite. The threads, strands, ribbons, or bands are multiple and are applied as a composite. The number of pieces of elastic material comprising the waist elastic 21 ranges from about 1 to about 20, preferably from about 2 to about 10, more preferably from 2 to about 8, and most preferably from about 2 to about 6. Preferably, when the waist elastic 21 are threads, 3 to 6 threads are used as the waist elastic 21, and the threads are spaced from about 0.0625 inch (1.6 mm) to about 1.5 inches (38 mm), more preferably from about 0.0625 inch (1.6 mm) to about 0.25 inch (6 mm), and most preferably about 0.125 inch (3 mm) apart.

The threads are made of elastomeric material. One suitable material is spandex such as LYCRA® threads available from DuPont located in Wilmington, Del. Suitable waist elastic 21 include threads having a decitex (g/10000 m) of from about 100 to about 1200, preferably from about 470 to about 940, and more preferably from about 620 to about 940 for waist elastic 21 comprising from about 5 to about 10 threads. Adhesive 71, applied in a meltblown or swirl pattern is used to bond the waist elastic 21 to the outer cover 13 and the body liner 80 or waist liner 26. Preferably, the adhesive 71 is applied only to the waist elastic 21. A suitable adhesive includes, for example, Findley H2096 hot melt adhesive which is available from Ato Findley Adhesives located in Milwaukee, Wis.

The leg elastics 32, 33, 34, and 35 are from about 0.0625 inch (1.6 mm) to about 1 inch (25 mm) wide, preferably from about 0.25 inch (6 mm) to about 1 inch (25 mm), and more preferably from about 0.25 inch (6 mm) to about 0.75 inch (18 mm). The leg elastics 32, 33, 34, and 35 are applied under an elongation of from about 100% to about 300%, preferably under an elongation of from about 175% to about 275%, and more preferably under an elongation of from about 200% to about 250%.

The leg elastics 32, 33, 34, and 35 include threads, strands, bands, ribbons, film, elastic nonwovens, or composite. The threads, strands, ribbons, or bands are multiple and are applied as a composite. The number of pieces of elastic material comprising the leg elastic 32, 33, 34, and 35 ranges from about 1 to about 6, preferably from about 2 to about 5, and more preferably from about 3 to about 4. Preferably, when the leg elastic 32, 33, 34, and 35 are threads, 1 to 6 threads are used as the leg elastic 32, 33, 34, and 35, and the threads are spaced from about 0.0625 inch (1.6 mm) to about 0.5 inches (13 mm), more preferably from about 0.0625 inch (1.6 mm) to about 0.25 inch (6 mm), and most preferably about 0.125 inch (3 mm) apart.

The threads are made of elastomeric material. One suitable material is spandex such as LYCRA® threads available from DuPont located in Wilmington, Del. Suitable leg elastics 32, 33, 34, and 35 include threads having a decitex (g/10000 m) of from about 470 to about 1200, preferably from about 620 to about 1000, and more preferably from about 740 to about 940 for leg elastics 32, 33, 34, and 35 having comprising from about 3 to about 6 threads. Adhesive 71 applied in a meltblown or swirl pattern is used to bond the leg elastics 32, 33, 34, and 35 to the outer cover 13 and to the body liner 80, the absorbent barrier composite 46, or the leg liner 38. Preferably, the adhesive 71 is applied only to the leg elastics 32, 33, 34, and 35. A suitable adhesive includes, for example, Findley H2096 hot melt adhesive which is available from Ato Findley Adhesives located in Milwaukee, Wis.

The crotch elastics 37 and 39 are from about 0.0625 inch (1.6 mm) to about 1 inch (25 mm) wide, preferably from about 0.25 inch (6 mm) to about 1 inch (25 mm), and more preferably from about 0.25 inch (6 mm) to about 0.75 inch (18 mm) such as 0.5 inch (13 mm). The crotch elastic 37 and 39 is applied under an elongation of from about 100% to about 300%, preferably under an elongation of from about 150% to about 275%, and more preferably under an elongation of from about 200% to about 250%.

The crotch elastics 37 and 39 include threads, strands, ribbons, bands, film, elastic nonwovens, or composite. The threads, strands, ribbons, or bands are multiple and are applied as a composite. The number of pieces of elastic material comprising the crotch elastic 37 and 39 ranges from about 1 to about 6, preferably from about 2 to about 5, and more preferably from about 3 to about 4. Preferably, when the crotch elastics 37 and 39 are threads, 1 to 6 threads are used as the crotch elastics 37 and 39, and the threads are spaced from about 0.0625 inch (1.6 mm) to about 0.5 inches (13 mm), more preferably from about 0.0625 inch (1.6 mm) to about 0.25 inch (6 mm), and most preferably about 0.125 inch (3 mm) apart.

The threads are made of elastomeric material. One suitable material is spandex such as LYCRAO threads available from DuPont located in Wilmington, Del. Suitable crotch elastics 37 and 39 include threads having a decitex (g/10000 m) of from about 470 to about 1200, preferably from about 620 to about 1000, and more preferably from about 740 to about 940 for crotch elastics 37 and 39 comprising from about 3 to about 6 threads. Adhesive 71 applied in a meltblown or swirl pattern is used to bond the crotch elastics 37 and 39 to the outer cover 13 and to the absorbent barrier composite 46 or the leg liner 38. Preferably the adhesive 71 is applied only to the crotch elastics 37 and 39. A suitable adhesive includes, for example, Findley H2096 hot melt adhesive which is available from Ato Findley Adhesives located in Milwaukee, Wis.

In one embodiment, a cradle structure is formed around the absorbent core 50 to provide a snug fit around the leg openings 28 and 30 and to draw up the sides of the crotch portion 18. The leg elastics 32, 33, 34, and 35 are applied to the outer cover 13 under an elongation of about 200% to about 250%. The crotch elastics 37 and 39 are sandwiched between the liquid barrier 48 and the topsheet layer 49 under an elongation of about 200%. Preferably, during the application of the elastics, the front leg elastics 32 and 33 and the back leg elastics 34 and 35 are elongated to a different degree and applied to the outer cover 13. In one embodiment, the front leg elastics 32 and 33 are elongated less than the back leg elastics 34 and 35.

In another embodiment providing a snug fit around the leg openings 28 and 30 and drawing the sides of the crotch portion 18 up to form a cradle structure around the absorbent core 50, the leg elastics 32, 33, 34, and 35 and the crotch elastics 37 and 39 are applied to the outer cover 13 under an elongation of about 200% to about 250%. Preferably, during the application of the elastics, the front leg elastics 32 and 33 and the back leg elastics 34 and 35 are elongated to a different degree and applied to the outer cover 13. In one embodiment, the front leg elastics 32 and 33 are elongated less than the back leg elastics 34 and 35.

In the embodiments having the three segment elastics surrounding each of the leg openings 28 and 30, the front leg elastics 32 and 33 and the crotch elastics 37 and 39 are elongated less than the back leg elastics 34 and 35. The three segment elastics include front leg elastics 32 and 33, back leg elastics 34 and 35, and crotch elastics 37 and 39. Preferably, the front leg elastics 32 and 33 and the crotch elastics 37 and 39 are elongated to about 200% and the back leg elastics 34 and 35 are elongated to about 250%. The three segment elastic system and differing tensions allow better fit, less tightness in the groin area, and less bunching of the crotch portion 18 caused by high leg elastic retraction. The back leg elastics 34 and 35 are under higher elongation to keep the seat of the underpant 12 from creeping up with movement during use and stabilizes the absorbent core 50 from bunching which causes leakage and an uncomfortable fit of the underpant 12.

In the embodiments having three segments of elastics surrounding each of the leg openings 28 and 30, the active portions of the crotch elastics 37 and 39 overlap with the active portions of the leg elastics 32, 33, 34, and 35 as shown in FIG. 1a showing such a configuration with the crotch elastic 39 and leg elastics 32 and 35. The active portions of the crotch and leg elastics 32, 33, 34, 35, 37, and 39 and the waist elastic 21 and the waist portion elastic 22 is the portion of the elastic exerting a contractive force on the article 10.

In FIG. 1a, another configuration is shown of the three segments of elastics surrounding each of the leg openings 28 and wherein the active portion of the crotch elastic 37 overlaps the active portion of the leg elastic 34 but does not overlap the active portion of the leg elastic 33. The configuration forms a gap 107 in the elastics surrounding the leg opening 30 in the front body portion 14. The gap 107 ranges in size from about 3 inches (76 mm) to about 0.125 inches (3 mm), preferably from about 2 inches (51 mm) to about 0.25 inch (6 mm), and more preferably from about 1 inch (25 mm) to about 0.5 inch (13 mm).

The gap 107 provides a more comfortable fit of the underpant 12. Binding or chafing are reduced from the leg elastics 32 and 33 and the crotch elastics 37 and 39 during use with certain body shapes and sizes and activities. The front lower body portion 42 is smoothed, providing a more discrete underpant 12.

Another configuration of the three segments of elastics surrounding each of the leg openings 28 and 30 is shown in FIG. 1b wherein the active portion of the crotch elastic 39 overlaps the active portion of the leg elastic 32 but does not overlap the active portion of the leg elastic 35. This configuration forms a gap 111 in the elastics surrounding the leg opening 28 in the 215 back body portion 15. The gap 111 ranges in size from about 3 inches (76 mm) to about 0.125 inches (3 mm), preferably from about 2 inches (51 mm) to about 0.25 inch (6 mm), and more preferably from about 1 inch (25 mm) to about 0.5 inch (13 mm).

The gap 111 provides a more comfortable fit of the underpant 12 in that binding and chafing are reduced that may result from the leg elastics 34 and 35 and the crotch elastics 37 and 39 during use with certain body shapes and sizes and activities. In addition, the back lower body portion 43 is smoothed, providing a more discrete underpant 12.

Another configuration of the three segments of elastics surrounding each of the leg openings 28 and 30 is shown in FIG. 1b wherein the active portion of the crotch elastic 37 does not overlap the active portion of the leg elastics 33 and 34. This configuration forms gaps 107 and 111 in the elastics surrounding the leg openings 28 and 30, respectively, in the front and back body portions 14 and 15 respectively. The gaps 107 and 111 each range in size from about 3 inches (76 mm) to about 0.125 inches (3 mm), preferably from about 2 inches (51 mm) to about 0.25 inch (6 mm), and more preferably from about 1 inch (25 mm) to about 0.5 inch (13 mm).

The gaps 107 and 111 provide a more comfortable fit of the underpant 12 in that binding or chafing that may result from the leg elastics 32, 33, 34, and 35 and the crotch elastics 37 and 39 during use with certain body shapes and sizes and activities are reduced. In addition, the back lower body portion 43 is smoothed, providing a more discrete underpant 12.

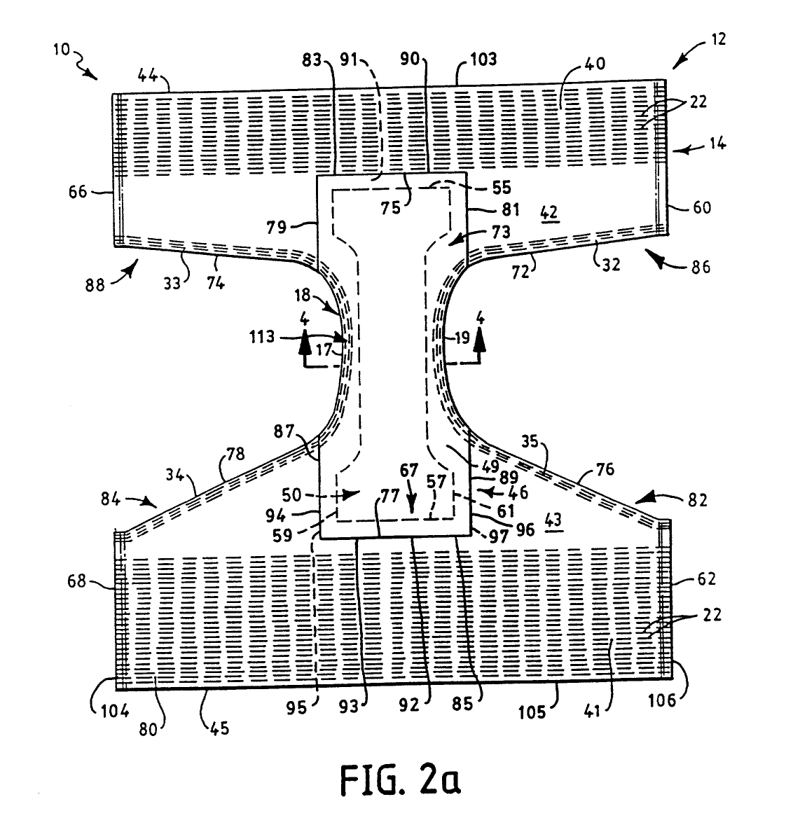

In the embodiment having the two segment elastics surrounding the leg openings 28 and 30, (see FIG. 2a), the front leg elastics 32 and 33 are elongated less than the back leg elastics 34 and 35. The two segment elastics include front leg elastics 32 and 33, and back leg elastics 34 and 35. Preferably, the front leg elastics 32 and 33 are elongated to about 200%, and the back leg elastics 34 and 35 are elongated to about 250%. The two segment elastic system and differing tensions provide better fit, less tightness in the groin area, and less bunching of the crotch portion 18 caused by high leg elastic retraction. The back leg elastics 34 and 35 are under higher elongation to help keep the seat of the underpant 12 from creeping up with movement during use and stabilizes the absorbent core 50 from bunching which causes leakage and an uncomfortable fit of the underpant 12.

Another configuration of the two segments of elastics surrounding each of the leg openings 28 and 30 is shown in FIG. 1c wherein the active portion of the leg elastic 32 does not overlap the active portion of the leg elastic 35. This configuration forms a gap 107 in the elastic surrounding the leg opening 28 at the top of the leg opening 28 in the front body portion 14, back body portion 15, or in both the front and back body portions 14 and 15. The gap 107 can range in size from about 3 inches (76 mm) to about 0.125 inches (3 mm), preferably from about 2 inches (51 mm) to about 0.25 inch (6 mm), and more preferably from about 1 inch (25 mm) to about 0.5 inch (13 mm). The gap 107 provides a more comfortable fit of the underpant 12 in that binding or chafing that may result from the leg elastics 32, 33, 34, and 35 during use with certain body shapes and sizes and activities are reduced. In addition, the gap 107 provides a more discrete underpant 12 as “panty lines” are eliminated at the top of the wearer's legs.

In the embodiments having two segments of elastics surrounding each of the leg openings 28 and 30, the active portions of the leg elastics 32 and 33 overlap with the active portions of the leg elastics 34 and 35 as shown in FIG. 2a showing such a configuration with the leg elastic 32 and leg elastic 35.

In FIG. 2a, another configuration of the two segments of elastics surrounding each of the leg openings 28 and 30 is shown wherein the active portion of the leg elastic 33 does not overlap the active portion of the leg elastic 34. This configuration forms a gap 113 in the elastics surrounding the leg opening 30 in the front body portion 14. The gap 113 can range in size from about 3 inches (76 mm) to about 0.125 inches (3 mm), preferably from about 2 inches (51 mm) to about 0.25 inch (6 mm) and more preferably from about 1 inch (25 mm) to about 0.5 inch (13 mm).

The gap 113 provides a more comfortable fit of the underpant 12 in that binding or chafing that may result from the leg elastics 32, 33, 34, and 35 during use with certain body shapes and sizes and activities are reduced.

Another configuration of the two segments of elastics surrounding each of the leg openings 28 and 30 is shown in FIG. 2a wherein the active portion of the leg elastic 32 does not overlap the active portion of the leg elastic 35. This configuration forms a gap 113 in the elastic surrounding the leg opening 28 at the top of the leg opening 28 in the front body portion 14, back body portion 15, or in both the front and back body portions 14 and 15. The gap 113 can range in size from about 3 inches (76 mm) to about 0.125 inches (3 mm), preferably from about 2 inches (51 mm) to about 0.25 inch (6 mm), and more preferably from about 1 inch (25 mm) to about 0.5 inch (13 mm). The gap 113 provides a more comfortable fit of the underpant 12 in that binding or chafing that may result from the leg elastics 32, 33, 34, and 35 during use with certain body shapes and sizes and activities are reduced. In addition, the gap 113 provides a more discrete underpant 12 as “panty lines” are eliminated at the top of the wearer's legs.

In the embodiment having a single segment elastic surrounding (see FIG. 3) the leg openings 28 and 30 (the single segment elastic include leg elastics 96 and 98), the leg elastics 96 and 98 are applied under an elongation of from about 100% to about 300%, preferably under an elongation of from about 175% to about 275%, and more preferably under an elongation of from about 200% to about 250%.

The waist portion elastic 22 circumferentially surrounding the body portions 14 and 15 of the underpant 12 act independently to conform to the contours of various body types and builds, providing a smooth, snug, and comfortable fit within a given hip size range. In embodiments having waist elastic 21, the use of higher elongation, closer spacing, and higher cross-sectional area in the waist elastic 21 than in the waist portion elastic 22, results in the underpant 12 having a rounded shape and good waist fit across a wide range the waist to hip ratios encountered. The elongation of the waist portion elastic 22 can be different in the front and back body portions 14 and 15.

Preferably, in the front body portion 14, the waist portion elastic 22 are spaced apart from the front leg elastics 32 and 33. The waist portion elastics 22 in the back body portion 15 are spaced apart from the back leg elastics 34 and 35. The distance between the waist portion elastic 22 and the leg elastics 32, 33, 34, and 35 ranges from about 0.125 inch (3 mm) to about 3 inches (76 mm), preferably from about 0.25 inch (6 mm) to about 2 inches (51 mm), and more preferably from about 0.25 inch (6 mm) to about 1.5 inches (38 mm). The spacing in the front body portions 14 and 15 are not required to be the same size. However, the waist portion elastic 22 in either or both of the front and back body portions adjoin the front or back leg elastics 32, 33, 34, and 35, respectively. In embodiments where waist elastic 21 are present, it is preferable that the waist portion elastic 22 are spaced apart from the waist elastic 21 and the leg elastics 32, 33, 34, and 35.

The waist portion elastic 22 in either or both of the front and back body portions may adjoin either or both the waist elastic 21 and the leg elastics 32, 33, 34, and 35 wherein the waist portion elastic 22 adjoin at least a portion of the leg elastics 32, 33, 34, and 35. In a underpant 12 sized comfortably to fit a hip range from about 36 inches (914 mm) to about 45 inches (1143 mm), the waist portion elastic 22 is about 4 inches (102 mm) wide in the front body portion 14 and about 6.25 inches (159 mm) wide in the back body portion 15. The waist portion elastic 22 is spaced from about 0.0625 inch (1.6 mm) to about 0.5 inch (13 mm) apart. Preferably, the waist portion elastic 22 is spaced from 0.0625 inch (1.6 mm) to about 0.25 inch (6 mm) apart. Most preferably, the waist portion elastic 22 is spaced about 0.125 inch (3 mm) apart.

The absorbent barrier composite 46 extending up the front and/or back body portions 14 and 15 toward the waist opening 20 is conformed to the wearer's body by the force exerted by the waist portion elastic 22. The transition from the front and back lower body portions 42 and 43 to the front and back upper body portions 40 and 41 thus is smoothed.

In embodiments having waist elastic 21, the waist elastic 21 preferably is under a greater tension per unit width than the waist portion elastic 22 in the upper body portions 40 and 41 to provide the snug waist fit over the range of waist to hip ratios of the various body shapes. In the preferred embodiment, the tension on the waist elastic 21 is coordinated with the tension of the waist portion elastic 22 to form a snug fit about the waist opening 20 while providing a smooth transition from the upper body portions 40 and 41 to the front and back waist edge portions 44 and 45. However, the tensions and the LYCRA® counts of the waist elastic 21 and the waist portion elastic 22 can be different.

In the front and back body portions 14 and 15, the leg liner 38, the waist liner 26, and/or the topsheet layer 49 are expanded to cover the interior of the body portions 14 and 15. In another embodiment, the leg liner 38, the waist liner 26, and/or the topsheet layer 49 exclude the center crotch portion 18 covered by the application of the absorbent barrier composite 46. In the body portions 14 and 15 where the absorbent barrier composite 46 overlaps the liner 80, the composite 46 is applied on top of any liner 80 present so as to contact the wearer. However, the overlapping portion of the absorbent barrier composite 46 is sandwiched between the outer cover 13 and the body liner 80.

For embodiments having waist portion elastic 22, leg elastics 32, 33, 34, and 35, and crotch elastics 37 and 39, the following ranges for the tensions of the elastics 22, 32, 33, 34, 35, 37, and 39 are provided in TABLE III below.

TABLE III

| | Acceptable | | More Acceptable | | Most Acceptable | |
|---|---|---|---|---|---|---|
| | Range | | Range | | Range | |
| | Lower | Upper | Lower | Upper | Lower | Upper |
| | Limit | Limit | Limit | Limit | Limit | Limit |
| Waist | 10 | 150 | 30 | 100 | 45 | 75 |
| Elastic | grams | grams | grams | grams | grams | grams |
| 22 | | | | | | |
| Leg | 20 | 400 | 80 | 220 | 120 | 180 |
| Elastics | grams | grams | grams | grams | grams | grams |
| 32, 33, | | | | | | |
| 34, and | | | | | | |
| 35 | | | | | | |
| Crotch | 20 | 400 | 80 | 220 | 120 | 180 |
| Elastics | grams | grams | grams | grams | grams | grams |
| 37 and | | | | | | |
| 39 | | | | | | |

For embodiments having waist elastic 21, waist portion elastic 22, leg elastics 32, 33, 34, and 35, and crotch elastics 37 and 39, the following ranges for the tensions of the elastics 21, 22, 32, 33, 34, 35, 37, and 39 are provided in TABLE IV below.

TABLE IV

| | Acceptable | | More Acceptable | | Most Acceptable | |
|---|---|---|---|---|---|---|
| | Range | | Range | | Range | |
| | Lower | Upper | Lower | Upper | Lower | Upper |
| | Limit | Limit | Limit | Limit | Limit | Limit |
| Waist | 30 | 300 | 50 | 200 | 100 | 175 |
| Elastic | grams | grams | grams | grams | grams | grams |
| 21 | | | | | | |
| Waist | 10 | 150 | 20 | 100 | 40 | 60 |
| Elastic | grams | grams | grams | grams | grams | grams |
| 22 | | | | | | |
| Leg | 20 | 400 | 80 | 220 | 120 | 180 |
| Elastics | grams | grams | grams | grams | grams | grams |
| 32, 33, | | | | | | |
| 34, and | | | | | | |
| 35 | | | | | | |
| Crotch | 20 | 400 | 80 | 220 | 120 | 180 |
| Elastics | grams | grams | grams | grams | grams | grams |
| 37 and | | | | | | |
| 39 | | | | | | |

The tensions of the waist elastic 21, waist portion elastic 22, crotch elastics 37 and 39, and leg elastics 32, 33, 34, and 35 were determined as follows. Samples having the dimensions of 10 mm wide and 51 mm in length were tested for each of the elastics 21, 22, 32, 33, 34, and 35.

The tensions stated in TABLE III and TABLE IV are given in grams per a 10 mm width. If the size of the sample being tested varies from a 10 mm width, the values must be normalized to a 10 mm width.

The tensions were measured during the first cycle extension at 100 percent elongation at room temperature and humidity.

The side seams 64 and 70 are made on the inside or outside of the underpant 12 or formed flat against the underpant 12 to give a more finished look to the underpant 12 and to prevent the side seams 64 and 70 from showing through clothing. Optionally, the lateral edges 60, 62, 66 and 68 of the front and back body portions are not overlapped but are formed flat and extend out laterally. The side seams 64 and 70 have minimal width while providing sufficient strength to be pulled up and down many times over a 24 hour wear period without tearing or breaking. Suitable side seams and seals are described in U.S. Pat. Nos. 4,610,681 issued Sep. 9, 1986, to Strohbeen et at., 4,641,381 issued Feb. 10, 1987 to Heran et al., and 4,646,362 issued Mar. 3, 1987 to Heran et al. which are incorporated herein by reference. In addition, the side seams 64 and 70 maybe refastenable. Various means of securing the disposable underpant 12 around the wearer include mechanical type fasteners. These include buttons and button holes, snaps, buckles, clasps, hooks and loops, end extensions, tabs, adhesive tapes which interlock or engage a complimentary device or the outer cover of the garment. In addition, elasticized fasteners are used in assuring better fit of the disposable underpant 12.

The side seams 64 and 70, respectively, of the outer cover 13 are sealed by ultrasonic bonding, stitching heat/pressure bonding, or adhesive bonding. The maximum seam strength attainable is dependent upon materials used, bond pattern, bond width, and process settings of dwell time, power, and pressure. Suitable side seams utilize ultrasonic bonding to achieve a seam strength such that the side seams 64 and 70 do not tear or open during use.

The side seams 64 and 70 have an unbonded portion outboard of the bonded area to provide for a soft edge to each of the side seams 64 and 70. The unbonded portion ranges from about 0 to about 13 mm in width, preferably from about 1 to about 6 mm in width and more preferably from about 2 to about 3 mm in width. Alternatively, the entire seam width bonded portion plus unbonded portion is less than about 0.6 inch (15 mm). If the seam is trimmed or cut close to the outer edge of the bond area, a sharp edge is produced along the seam edge which can catch on clothes or be irritating to the wearer's skin.

Having thus described the invention in detail, it will be readily apparent that various changes and modifications may be made without departing from the spirit of the invention. All such changes and modification are contemplated as being within the scope of the present invention, as defined by the following claims.

What is claimed is:

1. A disposable underpant defining an initial expanded shape having longitudinal and transverse axes, a front section, a back section, said front section and said back section being oppositely disposed on said longitudinal axis, and a central section disposed between said front section and said back section, each said section having a length along the longitudinal axis of one third of the length of said underpants comprising:
a) an outer cover having a front body portion and a back body portion connected by a crotch portion, said front and back body portions being connected together to form a waist opening and two leg openings; and
b) an absorbent barrier composite associated within at least said crotch portion having a liquid impervious layer, a pervious layer, and a rectangular absorbent core having a front end edge and a back end edge positioned between said liquid impervious layer and a pervious layer,
wherein said absorbent core is positioned within said sections such that the length of said absorbent core in the back section divided by the length of said absorbent core in said front section and said central section is less than 0.10 and said length of said absorbent core in said front section is greater than said length of absorbent core in said back section.

2. The disposable underpant of claim 1, wherein said length of said absorbent core in the back section divided by the length of said absorbent core in said front section and said central section is less than 0.095.

3. The disposable underpant of claim 2, wherein said length of said absorbent core in the back section divided by the length of said absorbent core in said front section and said central section is less than 0.085.

4. The disposable underpant of claim 3, wherein said length of said absorbent core in the back section divided by the length of said absorbent core in said front section and said central section is less than 0.07.

5. The disposable underpant of claim 1, wherein said outer cover further comprises leg elastic positioned around each of said leg openings to form a gather around each said leg opening, and waist portion elastic positioned around said front and back body portions to form gathers of said front and back body portions.

6. The disposable underpant of claim 2, wherein said outer cover further comprises leg elastic positioned around each of said leg openings to form a gather around each said leg opening, and waist portion elastic positioned around said front and back body portions to form gathers of said front and back body portions.

7. The disposable underpant of claim 1, wherein said outer cover further comprises waist elastic positioned around said waist opening to form gathers around said waist opening.

8. The disposable underpant of claim 7, wherein said outer cover further comprises waist elastic positioned around said waist opening to form gathers around said waist opening.

9. A disposable underpant defining an initial expanded shape having longitudinal and transverse axes, a front section, a back section, said front section and said back section being oppositely disposed on said longitudinal axis, and a central section disposed between said front section and said back section, each said section having a length along the longitudinal axis of one third of the length of said underpant, comprising:
a) an outer cover having a front body portion and a back body portion connected by a crotch portion, said front and back body portions being connected together to form a waist opening and two leg openings;
b) an absorbent barrier composite associated within at least said crotch portion having a liquid impervious layer, a pervious layer, and a rectangular absorbent core having a front end edge and a back end edge positioned between said liquid impervious layer and a pervious layer; and
c) said outer cover further comprising leg elastic positioned around each of said leg openings to form a gather around each said leg opening, and waist portion elastic positioned around said front and back body portions to form gathers of said front and back body portions,
wherein said absorbent core is positioned within said sections such that the length of said absorbent core in the back section divided by the length of said absorbent core in said front section and said central section is less than 0.10 and said length of said absorbent core in said front section is greater than said length of absorbent core in said back section.

10. The disposable underpant of claim 9, wherein said length of said absorbent core in the back section divided by the length of said absorbent core in said front section and said central section is less than 0.095.

11. The disposable underpant of claim 10, wherein said length of said absorbent core in the back section divided by the length of said absorbent core in said front section and said central section is less than 0.085.

12. The disposable underpant of claim 11, wherein said length of said absorbent core in the back section divided by the length of said absorbent core in said front section and said central section is less than 0.07.

13. The disposable underpant of claim 9, wherein said outer cover further comprises waist elastic positioned around said waist opening to form gathers around said waist opening.

14. The disposable underpant of claim 10, wherein said outer cover further comprises waist elastic positioned around said waist opening to form gathers around said waist opening.

15. A method of forming a disposable underpant defining an initial expanded shape having longitudinal and transverse axes, a front section, a back section, said front section and said back section being oppositely disposed on said longitudinal axis, and a central section disposed between said front section and said back section, each said section having a length along the longitudinal axis of one third of the length of said underpant, comprising:
a) providing an outer cover having a front body portion and a back body portion connected by a crotch portion, said front and back body portions connected together to form a waist opening and two leg openings; and
b) providing an absorbent barrier composite associated within at least said crotch portion having a liquid impervious layer, a pervious layer, and a rectangular absorbent core having a front end edge and a back end edge positioned between said liquid impervious layer and a pervious layer,
wherein said absorbent core is positioned within said sections such that the length of said absorbent core in the back section divided by the length of said absorbent core in said front section and said central section is less than 0.10 and said length of said absorbent core in said front section is greater than said length of absorbent core in said back section.

16. The method of forming a disposable underpant of claim 15, wherein said length of said absorbent core in the back section divided by the length of said absorbent core in said front section and said central section is less than 0.095.

17. The method of forming a disposable underpant of claim 16, wherein said length of said absorbent core in the back section divided by the length of said absorbent core in said front section and said central section is less than 0.085.

18. The method of forming a disposable underpant of claim 17, wherein said length of said absorbent core in the back section divided by the length of said absorbent core in said front section and said central section is less than 0.07.

19. The method of forming a disposable underpant of claim 15, further comprising positioning a waist elastic around said waist opening to form gathers around said waist opening.

20. The method of forming a disposable underpant of claim 8, further comprising positioning a waist elastic around said waist opening to form gathers around said waist opening.
